SEQUENCE LISTING (45 sequences)

SEQ ID NO: 1 - DNA, 15 bp, synthetic construct
  source 1..15: mol_type = other DNA
ggctagctacaacga

SEQ ID NO: 2 - DNA, 13 bp, synthetic construct
  source 1..13: mol_type = other DNA
ccgagccggacga

SEQ ID NO: 3 - DNA, 14 bp, synthetic construct
  source 1..14: mol_type = other DNA
gtcagcgacacgaa

SEQ ID NO: 4 - DNA, 14 bp, synthetic construct
  source 1..14: mol_type = other DNA
gtcagtgactcgaa

SEQ ID NO: 5 - DNA, 15 bp, synthetic construct
  source 1..15: mol_type = other DNA
gtcagctgactcgaa

SEQ ID NO: 6 - DNA, 20 bp, synthetic construct
  source 1..20: mol_type = other DNA
aggaggtaggggttccgctc

SEQ ID NO: 7-12 - skipped (6 entries)

SEQ ID NO: 13 - DNA, 33 bp, synthetic construct
  source 1..33: mol_type = other DNA
  modified_base 33: mod_base = OTHER; note = Guanine 3' Cholesterol-triethylene glycol
gatcaggtgggctagctacaacgaaacggcagg

SEQ ID NO: 14 - DNA, 31 bp, synthetic construct
  source 1..31: mol_type = other DNA
  modified_base 31: mod_base = OTHER; note = Guanine 3' Cholesterol-triethylene glycol
gatcaggtgccgagccggacgaaacggcagg

SEQ ID NO: 15 - DNA, 32 bp, synthetic construct
  source 1..32: mol_type = other DNA
  modified_base 32: mod_base = OTHER; note = Guanine 3' Cholesterol-triethylene glycol
gatcaggtggtcagcgacacgaaaacggcagg

SEQ ID NO: 16 - DNA, 32 bp, synthetic construct
  source 1..32: mol_type = other DNA
  modified_base 32: mod_base = OTHER; note = Guanine 3' Cholesterol-triethylene glycol
gatcaggtggtcagtgactcgaaaacggcagg

SEQ ID NO: 17 - DNA, 33 bp, synthetic construct
  source 1..33: mol_type = other DNA
  modified_base 33: mod_base = OTHER; note = Guanine 3' Cholesterol-triethylene glycol
gatcaggtggtcagctgactcgaaaacggcagg

SEQ ID NO: 18 - DNA, 38 bp, synthetic construct
  source 1..38: mol_type = other DNA
  modified_base 38: mod_base = OTHER; note = Guanine 3' Cholesterol-triethylene glycol
gatcaggtgaggaggtaggggttccgctcaacggcagg

SEQ ID NO: 19 - DNA, 33 bp, synthetic construct
  source 1..33: mol_type = other DNA
  modified_base 33: mod_base = OTHER; note = Guanine 3' Cholesterol-triethylene glycol
cttgaccagggctagctacaacgagacttcagg

SEQ ID NO: 20 - DNA, 31 bp, synthetic construct
  source 1..31: mol_type = other DNA
  modified_base 31: mod_base = OTHER; note = Guanine 3' Cholesterol-triethylene glycol
cttgaccagccgagccggacgagacttcagg

SEQ ID NO: 21 - DNA, 32 bp, synthetic construct
  source 1..32: mol_type = other DNA
  modified_base 32: mod_base = OTHER; note = Guanine 3' Cholesterol-triethylene glycol
cttgaccaggtcagcgacacgaagacttcagg

SEQ ID NO: 22 - DNA, 32 bp, synthetic construct
  source 1..32: mol_type = other DNA
  modified_base 32: mod_base = OTHER; note = Guanine 3' Cholesterol-triethylene glycol
cttgaccaggtcagtgactcgaagacttcagg

SEQ ID NO: 23 - DNA, 33 bp, synthetic construct
  source 1..33: mol_type = other DNA
  modified_base 33: mod_base = OTHER; note = Guanine 3' Cholesterol-triethylene glycol
cttgaccaggtcagctgactcgaagacttcagg

SEQ ID NO: 24 - DNA, 38 bp, synthetic construct
  source 1..38: mol_type = other DNA
  modified_base 38: mod_base = OTHER; note = Guanine 3' Cholesterol-triethylene glycol
cttgaccagaggaggtaggggttccgctcgacttcagg

SEQ ID NO: 25 - DNA, 33 bp, synthetic construct
  source 1..33: mol_type = other DNA
  modified_base 33: mod_base = OTHER; note = Cytosine 3' Cholesterol-triethylene glycol
atctcggcaggctagctacaacgagctgacgac

SEQ ID NO: 26 - DNA, 31 bp, synthetic construct
  source 1..31: mol_type = other DNA
  modified_base 31: mod_base = OTHER; note = Cytosine 3' Cholesterol-triethylene glycol
atctcggcaccgagccggacgagctgacgac

SEQ ID NO: 27 - DNA, 32 bp, synthetic construct
  source 1..32: mol_type = other DNA
  modified_base 32: mod_base = OTHER; note = Cytosine 3' Cholesterol-triethylene glycol
atctcggcagtcagcgacacgaagctgacgac

SEQ ID NO: 28 - DNA, 32 bp, synthetic construct
  source 1..32: mol_type = other DNA
  modified_base 32: mod_base = OTHER; note = Cytosine 3' Cholesterol-triethylene glycol
atctcggcagtcagtgactcgaagctgacgac

SEQ ID NO: 29 - DNA, 33 bp, synthetic construct
  source 1..33: mol_type = other DNA
  modified_base 33: mod_base = OTHER; note = Cytosine 3' Cholesterol-triethylene glycol
atctcggcagtcagctgactcgaagctgacgac

SEQ ID NO: 30 - DNA, 38 bp, synthetic construct
  source 1..38: mol_type = other DNA
  modified_base 38: mod_base = OTHER; note = Cytosine 3' Cholesterol-triethylene glycol
atctcggcaaggaggtaggggttccgctcgctgacgac

SEQ ID NO: 31-32 - skipped (2 entries)

SEQ ID NO: 33 - DNA, 33 bp, synthetic construct
  source 1..33: mol_type = other DNA
  modified_base 33: mod_base = OTHER; note = Cytosine 3' Cholesterol-triethylene glycol
ggcgttctgggctagctacaacgatcgtggatc

SEQ ID NO: 34 - DNA, 31 bp, synthetic construct
  source 1..31: mol_type = other DNA
  modified_base 31: mod_base = OTHER; note = Cytosine 3' Cholesterol-triethylene glycol
ggcgttctgccgagccggacgatcgtggatc

SEQ ID NO: 35 - DNA, 32 bp, synthetic construct
  source 1..32: mol_type = other DNA
  modified_base 32: mod_base = OTHER; note = Cytosine 3' Cholesterol-triethylene glycol
ggcgttctggtcagcgacacgaatcgtggatc

SEQ ID NO: 36 - DNA, 32 bp, synthetic construct
  source 1..32: mol_type = other DNA
  modified_base 32: mod_base = OTHER; note = Cytosine 3' Cholesterol-triethylene glycol
ggcgttctggtcagtgactcgaatcgtggatc

SEQ ID NO: 37 - DNA, 33 bp, synthetic construct
  source 1..33: mol_type = other DNA
  modified_base 33: mod_base = OTHER; note = Cytosine 3' Cholesterol-triethylene glycol
ggcgttctggtcagctgactcgaatcgtggatc

SEQ ID NO: 38 - DNA, 38 bp, synthetic construct
  source 1..38: mol_type = other DNA
  modified_base 38: mod_base = OTHER; note = Cytosine 3' Cholesterol-triethylene glycol
ggcgttctgaggaggtaggggttccgctctcgtggatc

SEQ ID NO: 39 - DNA, 2793 bp, Escherichia coli
  source 1..2793: mol_type = genomic DNA
gtcgacagccatcatgatgctgttgcgtattgattatgaaacgcgtctggagaaagcgcaggcgtttgtacgaggttcacgatgagtggtcaaggaaagcgattaatggtgatggcaggcggaaccggtggacatgtattcccgggactggcggttgcgcaccatctaatggctcagggttggcaagttcgctggctggggactgccgaccgtatggaagcggacttagtgccaaaacatggcatcgaaattgatttcattcgtatctctggtctgcgtggaaaaggtataaaagcactgatagctgccccgctgcgtatcttcaacgcctggcgtcaggcgcgggcgattatgaaagcgtacaaacctgacgtggtgctcggtatgggaggctacgtgtcaggtccaggtggtctggccgcgtggtcgttaggcattccggttgtacttcatgaacaaaacggtattgcgggcttaaccaataaatggctggcgaagattgccaccaaagtgatgcaggcgtttccaggtgctttccctaatgcggaagtagtgggtaacccggtgcgtaccgatgtgttggcgctgccgttgccgcagcaacgtttggctggacgtgaaggtccggttcgtgtgctggtagtgggtggttctcagggcgcacgcattcttaaccagacaatgccgcaggttgctgcgaaactgggtgattcagtcactatctggcatcagagcggcaaaggttcgcaacaatccgttgaacaggcgtatgccgaagcggggcaaccgcagcataaagtgacggaatttattgatgatatggcggcggcgtatgcgtgggcggatgtcgtcgtttgccgctccggtgcgttaacggtgagtgaaatcgccgcggcaggactaccggcgttgtttgtgccgtttcaacataaagaccgccagcaatactggaatgcgctaccgctggaaaaagcgggcgcagccaaaattatcgagcagccacagcttagcgtggatgctgtcgccaacaccctggccgggtggtcgcgagaaaccttattaaccatggcagaacgcgcccgcgctgcatccattccggatgccaccgagcgagtggcaaatgaagtgagccgggttgcccgggcgtaattgtagcgatgccttttgcatcgtatgaatttaagaagttaatggcgtaaagaatgaatacacaacaattggcaaaactgcgttccatcgtgcccgaaatgcgtcgcgttcggcacatacattttgtcggcattggtggtgccggtatgggcggtattgccgaagttctggccaatgaaggttatcagatcagtggttccgatttagcgccaaatccggtcacgcagcagttaatgaatctgggtgcgacgatttatttcaaccatcgcccggaaaacgtacgtgatgccagcgtggtcgttgtttccagcgcgatttctgccgataacccggaaattgtcgccgctcatgaagcgcgtattccggtgatccgtcgtgccgaaatgctggctgagttaatgcgttttcgtcatggcatcgccattgccggaacgcacggcaaaacgacaaccaccgcgatggtttccagcatctacgcagaagcggggctcgacccaaccttcgttaacggcgggctggtaaaagcggcgggggttcatgcgcgtttggggcatggtcggtacctgattgccgaagcagatgagagtgatgcatcgttcctgcatctgcaaccgatggtggcgattgtcaccaatatcgaagccgaccacatggatacctaccagggcgactttgagaatttaaaacagacttttattaattttctgcacaacctgccgttttacggtcgtgcggtgatgtgtgttgatgatccggtgatccgcgaattgttaccgcgagtggggcgtcagaccacgacttacggcttcagcgaagatgccgacgtgcgtgtagaagattatcagcagattggcccgcaggggcactttacgctgctgcgccaggacaaagagccgatgcgcgtcaccctgaatgcgccaggtcgtcataacgcgctgaacgccgcagctgcggttgcggttgctacggaagagggcattgacgacgaggctattttgcgggcgcttgaaagcttccaggggactggtcgccgttttgatttcctcggtgaattcccgctggagccagtgaatggtaaaagcggtacggcaatgctggtcgatgactacggccaccacccgacggaagtggacgccaccattaaagcggcgcgcgcaggctggccggataaaaacctggtaatgctgtttcagccgcaccgttttacccgtacgcgcgacctgtatgatgatttcgccaatgtgctgacgcaggttgataccctgttgatgctggaagtgtatccggctggcgaagcgccaattccgggagcggacagccgttcgctgtgtcgcacaattcgtggacgtgggaaaattgatcccattctggtgccggatccggcgcgggtagccgagatgctggcaccggtattaaccggtaacgacctgattctcgttcagggggctggtaatattggaaaaattgcccgttctttagctgaaatcaaactgaagccgcaaactccggaggaagaacaacatgactgataaaatcgcggtcctgttgggtgggacctccgctgagcgggaagtttctctgaattctggcgcagcggtgttagccggactgcgtgaaggcggtattgacgcgtatcctgtcgac

SEQ ID NO: 40 - DNA, 1083 bp, Streptococcus pyogenes
  source 1..1083: mol_type = genomic DNA
atgcctaagaagattttatttacaggtggtggaactgtaggtcatgtcaccttgaacctcattctcataccaaaatttatcaaggacggttgggaagtacattatattggtgataaaaatggcattgaacatacagaaattgaaaagtcaggccttgacgtgacctttcatgctatcgcgacaggcaagcttagacgctatttttcatggcaaaatctagctgatgtttttaaggttgcacttggcctcctacagtctctctttattgttgccaagcttcgccctcaagcccttttttccaaaggtggttttgtctcagtaccgccagttgtggctgctaaattgcttggtaaaccagtctttattcatgaatcagatcggtcaatgggactagcaaacaagattgcctacaaatttgcaactaccgtgtataccacttttgagcaggaagaccagttgtctaaagttaaacaccttggagcggtgacaaaggttttcaaagatgccaaccaaatacctgaatcaactcagttagaggcggtgaaagagtattttagtagagacctaaaaaccctcttgtttattggtggttcggcaggggcgcatgtgtttaatcagtttattagtgatcatccagaattgaagcaacgttataatatcatcaatattacaggagaccctcaccttaatgaattgagttctcatctgtatcgagtagattatgttaccgatctctaccaacctttgatggcgatggctgaccttgtagtgacaagagggggctctaatacactttttgagctactggcaatggctaagctacacctcatcgttcctcttggtaaagaagctagccgtggcgatcagttagaaaatgccacttattttgagaagaggggctacgctaaacaattacaggaacctgatttaactttgcataattttgatcaggcaatggctgatttgtttgaacatcaggctgattatgaggctactatgttggcaactaaggagattcaatcaccggacttcttttatgaccttttgagagctgatattagctccgcgattaaggagaagtaa

SEQ ID NO: 41 - DNA, 1071 bp, Staphylococcus aureus
  source 1..1071: mol_type = genomic DNA
atgacgaaaatcgcatttaccggagggggaacagttggacacgtatcagtaaatttaagtttaattccaactgcattatcacaaggatatgaagcgctttatattggttctaaaaatggtattgaaagagaaatgattgaatcacaactaccagaaattaagtattatcctatttcgagtggtaaattaacaagatatatttctttagaaaatgccaaaaacgtatttaaagtattgaaaggtattcttgatgctcgtaaagttttgaaaaaagaaaaacctgatctattattttcaaaaggtggatttgtatctgtgcctgttgttattgcagccaaatcattaaatataccaactattattcatgaatctgacttaacaccaggattagcgaataagatagcacttaaatttgccaagaaaatatatacaacatttgaagaaacgctaaactacttacctaaagagaaagctgattttattggagcaacaattcgagaagatttaaataatggtaatgcacataatggttatcaattaacaggctttaatgaaaataaaaaagttttactcgttatgggtggaagcttaggaagtaaaaaattaaatagcattattcgcgaaaacttagatgcattattacaacaatatcaagtgatacatttaactggtaaaggattaaaagatgctcaagttaaaaaatcaggatatatacaatatgaatttgttaaagaggatttaacagatttattagcaattacggatacagtaataagtagagctggatcaaatgcgatttatgagttcttaacattacgtataccaatgttattagtaccattaggtttagatcaatcccgaggcgaccaaattgacaatgcaaatcattttgctgataaaggatatgctaaagcgattgatgaagaacaattaacagcacaaattttattacaagaactaaatgaaatggtacaggaaacaactcgaattatcaataatatgaaatcgtatgaacaaagttatacgaaagaagctttatttgataagatgattaaagacgcattgaattaa

SEQ ID NO: 42 - skipped

SEQ ID NO: 43 - DNA, 35 bp, synthetic construct
  source 1..35: mol_type = other DNA
  misc_difference 1: note = a, g, c, or t
  misc_difference 2: note = a, g, c, or t
  misc_difference 3: note = a, g, c, or t
  misc_difference 4: note = a, g, c, or t
  misc_difference 5: note = a, g, c, or t
  misc_difference 6: note = a, g, c, or t
  misc_difference 7: note = a, g, c, or t
  misc_difference 8: note = a, g, c, or t
  misc_difference 9: note = a, g, c, or t
  misc_difference 26: note = a, g, c, or t
  misc_difference 27: note = a, g, c, or t
  misc_difference 28: note = a, g, c, or t
  misc_difference 29: note = a, g, c, or t
  misc_difference 30: note = a, g, c, or t
  misc_difference 31: note = a, g, c, or t
  misc_difference 32: note = a, g, c, or t
  misc_difference 33: note = a, g, c, or t
  misc_difference 34: note = a, g, c, or t
  misc_difference 35: note = a, g, c, or t
nnnnnnnnnrggctagctacaacgannnnnnnnnn

SEQ ID NO: 44 - DNA, 33 bp, synthetic construct
  source 1..33: mol_type = other DNA
  modified_base 33: mod_base = OTHER; note = Adenine 3' Cholesterol-triethylene glycol
accacaataccgagatcggtcgtgtcggcatga

SEQ ID NO: 45 - DNA, 1074 bp, Pseudomonas aeruginosa
  source 1..1074: mol_type = genomic DNA
atgaaaggtaatgtcctgatcatggcgggtggcaccggcggacacgtgttcccggcgctcgcctgtgcgcgggagttccaggcgcgcggctatgccgtgcactggctggggacgccgcgcggcatcgagaatgacctggtacccaaggccggcctgccgttgcacctgatccaggtcagcgggctgcgcggcaagggcctgaagtcgctggtcaaggcgccgctggaactgctcaagtcgctgttccaggcgctgcggctgatccgccagctgaggccggtctgcgtgctcggcctgggtggctatgtcaccggcccgggcggcctggccgcgcgcctcaacggcgtgccgctggtgatccacgagcagaacgccgtggccggcaccgccaaccgcagcctggcgccgatcgccaggcgcgtctgcgaggcattcccggataccttcccggccagcgacaagcgtctgaccaccggcaatccggtgcgcgccgagctgttcctcgacgcgcatgcccgcgcgccgctgaccggccgtcgggtcaatctgctggtgctcggcggcagccttggcgcggaaccgctgaacaagctgttgccagaggccctggcgcaggtgccgctggaaatccgtccggcaatccgccatcaggccggtcgtcagcatgccgagattaccgccgagcgttatcgcacagtggcggtcgaagcggacgtcgcgcccttcatcagcgacatggccgcggcctatgcctgggccgacctggtgatctgtcgcgccggcgcgctgaccgtcagcgagctgacggcggcgggtttgcccgccttcctggtgccgttgcctcacgcgatcgacgatcaccagacccgcaatgccgaattcctggtacgcagcggcgccggccgcctgctgccgcaaaagtctaccggcgcggccgaactggccgcgcagctgtccgaggtcctgatgcatcccgaaaccctgcgctccatggcagaccaggcacgcagcctggcgaaacccgaggctacccggacggtggtcgatgcctgcctggaggtggcccgtggttaa

(19) United States
(12) Patent Application Publication
(10) Pub. No.: US 2024/0336923 A1
(43) Pub. Date: Oct. 10, 2024

(54) DNAZYMES TARGETING CELL WALL SYNTHESIS ENZYMES AND USES THEREOF

(71) Applicant: 1E Therapeutics, Ltd., Rehovot (IL)

(72) Inventors: Ido BACHELET, Rehovot (IL); Almogit ABU-HOROWITZ, Rehovot (IL); Alexander ROSENBERG, Rehovot (IL); Ron OSHRI, Rehovot (IL); Adva LEVY-ZAMIR, Rehovot (IL); Ilana KOLODKIN-GAL, Rehovot (IL); Ella GILLIS, Rehovot (IL)

(73) Assignee: 1E Therapeutics, Ltd., Rehovot (IL)

(21) Appl. No.: 18/682,542

(22) PCT Filed: Aug. 11, 2022
(86) PCT No.: PCT/IL2022/000001

§ 371 Date: Feb. 9, 2024

Related U.S. Application Data
(60) Provisional application No. 63/232,992, filed on Aug. 13, 2021.

(51) Int. Cl. C12N 15/113; A61K 31/7125; A61K 31/713; A61P 31/04

(52) U.S. Cl. CPC C12N 15/1137; A61K 31/7125; A61K 31/713; A61P 31/04; C12N 2310/127; C12N 2310/3515

(57) ABSTRACT

Provided herein are compositions and methods comprising a DNAzyme targeting a transcript encoding a cell wall synthesis enzyme, for use in reducing an amount of a biofilm in a subject with a bacterial infection; increasing or enhancing antibiotic susceptibility in a subject with a bacterial infection; and inhibiting bacterial growth in a subject with a bacterial infection.

### BACKGROUND

The bacterial gene murG is an UDP-N-acetylglucosamine-N-acetylmuramyl-(pentapeptide) pyrophosphoryl-undecaprenol N-acetylglucosamine transferase that catalyzes the synthesis of the GlcNac polysaccharides of the bacterial envelope. Non-limiting examples of murG sequences are the Pseudomonas aeruginosa murG (available at the world wide web uniprot.org/uniprot/Q9HWO1); and murG genes from Escherichia coli, Bacillus subtilis, Enterococcus faecium, Streptococcus pyogenes, Staphylococcus aureus, Klebsiella pneumonia, Acinetobacter baumannii, and Enterobacter cloacae. murG is highly conserved—see, for example, FIGS. 1B-1F, illustrating its conservation in P. aeruginosa isolates.

Antibiotic-resistant bacteria are a critical global health challenge. A recent development in antibacterial treatment is antibacterial antisense oligonucleotides. This is generally described as RNA silencing in bacteria using synthetic nucleic acid oligomer mimetics to specifically inhibit essential gene expression and achieve gene-specific antibacterial effects. Usually the antibacterial antisense oligonucleotides are designed to bind the target RNA transcript to prevent translation or bind DNA to prevent gene transcription respectively (Bai and Luo., A Search for Antibacterial Agents, 2012, chapter 16: 319-344; ISBN 978-953-51-0724-8, InTech, Chapters). However, there remains a clear unmet need for the development of additional techniques exploiting novel antibacterial mechanisms.

### SUMMARY

In certain aspects, provided herein are compositions and methods including DNAzymes targeting a transcript encoding a cell wall synthesis enzyme, as well as nucleic acids and vectors encoding such DNAzymes and pharmaceutical compositions comprising such DNAzymes. In some aspects, provided herein are methods of using such DNAzymes, nucleic acids, vectors and/or pharmaceutical compositions for treating and/or preventing bacterial infections, inhibiting bacterial growth, inhibiting biofilm formation, and inducing death of bacteria. In related aspects, the cell wall synthesis enzyme is encoded by murG.

In certain embodiments, there is a provided a method of reducing an amount of a biofilm in a subject with a bacterial infection, comprising administering to the subject a DNAzyme targeting a transcript encoding a cell wall synthesis enzyme, thereby reducing an amount of a biofilm in a subject. In some embodiments, the bacterial strain is an antibiotic-resistant bacterial strain.

In other embodiments, there is a provided a method of inhibiting formation of a biofilm in a subject with a bacterial infection, comprising administering to the subject a DNAzyme targeting a transcript encoding a cell wall synthesis enzyme, thereby inhibiting formation of a biofilm in a subject. In some embodiments, the bacterial strain is an antibiotic-resistant bacterial strain.

In still other embodiments, there is a provided a method of increasing antibiotic susceptibility in a subject with a bacterial infection, comprising administering to the subject a DNAzyme targeting a transcript encoding a cell wall synthesis enzyme, thereby increasing antibiotic susceptibility in a subject. In some embodiments, the bacteria are in a biofilm. Alternatively or in addition, the bacterial strain is an antibiotic-resistant bacterial strain.

In still other embodiments, there is a provided a method of enhancing antibiotic effectiveness in a subject with a bacterial infection, comprising administering to the subject a DNAzyme targeting a transcript encoding a cell wall synthesis enzyme, thereby enhancing antibiotic effectiveness in a subject. In some embodiments, the bacteria are in a biofilm. Alternatively or in addition, the bacterial strain is an antibiotic-resistant bacterial strain.

In yet other embodiments, there is a provided a method of inhibiting growth of a bacterium, comprising contacting the bacteria with a DNAzyme targeting a murG RNA transcript, wherein, upon binding of the DNAzyme to the murG RNA transcript, the DNAzyme cleaves the murG RNA transcript, thereby inhibiting growth of a bacterium. As provided herein, the described DNAzymes are capable of inhibiting growth of bacterial populations. In some embodiments, the bacteria are in a biofilm. Alternatively or in addition, the bacterial strain is an antibiotic-resistant bacterial strain. In a related aspect, the bacteria is brought into contact with the DNAzyme by treating a bacteria-infected cell with the DNAzyme. In a further related aspect, the bacteria is brought into contact with the DNAzyme by treating a bacteria-infected subject with the DNAzyme.

In other aspects, provided herein are DNAzymes targeting murG RNA transcript, as well as nucleic acids and vectors encoding such DNAzymes, and pharmaceutical compositions comprising such DNAzymes. In some aspects, provided herein are methods of using such DNAzymes, nucleic acids, vectors and/or pharmaceutical compositions for treating and/or preventing bacterial infections, inhibiting bacterial growth, reducing biofilm mass, and inducing death of bacteria.

In certain aspects, provided herein are DNAzymes targeting a murG RNA transcript, the DNAzyme comprising, in 5′ to 3′ order: (i) a first substrate-binding domain (also referred to herein as the “5′ arm”) comprising a sequence that base pairs with a first region of the murG RNA transcript; (ii) a DNAzyme catalytic core; and (iii) a second substrate-binding domain (also referred to herein as the “3′ arm”) comprising a sequence that base pairs with a second region of the murG RNA transcript positioned 5′ to the first region of the murG RNA transcript, wherein upon binding of the DNAzyme to the murG RNA transcript, the DNAzyme catalytic core cleaves the murG RNA transcript at a position between the first and second region of the murG RNA transcript.

In a related aspect, the DNAzyme catalytic core is a 10-23 catalytic core, an 8-17 catalytic core, a E1111 catalytic core, a E2112 catalytic core, a E5112 catalytic core, or a bipartite catalytic core. In further related aspects, the DNAzyme catalytic core is a 10-23 catalytic core. another further related aspect, the DNAzyme catalytic core comprises a nucleic acid sequence selected from any one of SEQ ID NOs: 1-6. In another further related aspect, the DNAzyme catalytic core comprises the nucleic acid sequence of SEQ ID NO: 1.

In a related aspect, the 5′ arm is any of the lengths or length ranges mentioned herein. In another related aspect, the 3′ arm is any of the lengths or length ranges mentioned herein. In yet another related aspect, the 5′ arm and the 3′ arm are independently selected from any of the lengths or length ranges mentioned herein. In further related aspects, the first substrate-binding domain and the second substrate-binding domain are 6-15 nucleotides in length.

In a related aspect, the 5′ arm is fully complementary (100%) to the first region of the RNA transcript or partially complementary to the first region of the RNA transcript with no more than two mismatches. In another related aspect, the 3′ arm is fully complementary (100%) to the second region of the RNA transcript or partially complementary to the second region of the RNA transcript with no more than two mismatches. In another related aspect, the 5′ arm and the 3′ arm together have no more than 3 mismatches to the first and second regions of the RNA transcript.

In some embodiments, the first substrate-binding domain is fully complementary (100%) to the first region of the murG RNA transcript or partially complementary to the first region of the murG RNA transcript with no more than two mismatches. In some embodiments, the second substrate-binding domain is fully complementary (100%) to the second region of the murG RNA transcript or partially complementary to the second region of the murG RNA transcript with no more than two mismatches. In some embodiments, the first substrate-binding domain and the second substrate-binding domain together have no more than 3 mismatches to the first and second regions of the murG RNA transcript.

In some embodiments, the nucleic acid sequence of the first substrate-binding domain comprises 5′-GATCAGGTG-3′ (SEQ ID NO: 7), and sequence of the second substrate-binding domain comprises 5′-AACGGCAGG-3′ (SEQ ID NO: 8). In other embodiments, the sequence of the first substrate-binding domain consists of SEQ ID NO: 7. In other embodiments, the sequence of the second substrate-binding domain consists of SEQ ID NO: 8. In still other embodiments, the sequence of the first substrate-binding domain consists of SEQ ID NO: 7, and the sequence of the second substrate-binding domain consists of SEQ ID NO: 8.

In some embodiments, the sequence of the DNAzyme comprises a sequence selected from SEQ ID NOs: 13-18. In certain embodiments, the sequence of the DNAzyme comprises SEQ ID NO: 13. In other embodiments, the sequence of the DNAzyme consists of a nucleic acid sequence selected from SEQ ID NOs: 13-18. In certain embodiments, the sequence of the DNAzyme consists of SEQ ID NO: 13.

In some embodiments, the nucleic acid sequence of the first substrate-binding domain comprises 5′-CTTGACCAG-3′ (SEQ ID NO: 9), and sequence of the second substrate-binding domain comprises 5′-GACTTCAGG-3′ (SEQ ID NO: 10). In other embodiments, the sequence of the first substrate-binding domain consists of SEQ ID NO: 9. In other embodiments, the sequence of the second substrate-binding domain consists of SEQ ID NO: 10. In still other embodiments, the sequence of the first substrate-binding domain consists of SEQ ID NO: 9, and the sequence of the second substrate-binding domain consists of SEQ ID NO: 10.

In some embodiments, the sequence of the DNAzyme comprises a sequence selected from SEQ ID NOs: 19-24. In certain embodiments, the sequence of the DNAzyme comprises SEQ ID NO: 19. In other embodiments, the sequence of the DNAzyme consists of a nucleic acid sequence selected from SEQ ID NOs: 19-24. In certain embodiments, the sequence of the DNAzyme consists of SEQ ID NO: 19.

In some embodiments, the nucleic acid sequence of the first substrate-binding domain comprises 5′-ATCTCGGCA-3′ (SEQ ID NO: 11), and sequence of the second substrate-binding domain comprises 5′-GCTGACGAC-3′ (SEQ ID NO: 12). In other embodiments, the sequence of the first substrate-binding domain consists of SEQ ID NO: 11. In other embodiments, the sequence of the second substrate-binding domain consists of SEQ ID NO: 12. In still other embodiments, the sequence of the first substrate-binding domain consists of SEQ ID NO: 11, and the sequence of the second substrate-binding domain consists of SEQ ID NO: 12.

In some embodiments, the sequence of the DNAzyme comprises a sequence selected from SEQ ID NOs: 25-30. In certain embodiments, the sequence of the DNAzyme comprises SEQ ID NO: 25. In other embodiments, the sequence of the DNAzyme consists of a nucleic acid sequence selected from SEQ ID NOs: 25-30. In certain embodiments, the sequence of the DNAzyme consists of SEQ ID NO: 25.

In some embodiments, the nucleic acid sequence of the first substrate-binding domain comprises 5′-GGCGTTCTG-3′ (SEQ ID NO: 31), and sequence of the second substrate-binding domain comprises 5′-TCGTGGATC-3′ (SEQ ID NO: 32). In other embodiments, the sequence of the first substrate-binding domain consists of SEQ ID NO: 31. In other embodiments, the sequence of the second substrate-binding domain consists of SEQ ID NO: 32. In still other embodiments, the sequence of the first substrate-binding domain consists of SEQ ID NO: 31, and the sequence of the second substrate-binding domain consists of SEQ ID NO: 32.

In some embodiments, the sequence of the DNAzyme comprises a sequence selected from SEQ ID NOs: 33-38. In certain embodiments, the sequence of the DNAzyme comprises SEQ ID NO: 33. In other embodiments, the sequence of the DNAzyme consists of a nucleic acid sequence selected from SEQ ID NOs: 33-38. In certain embodiments, the sequence of the DNAzyme consists of SEQ ID NO: 33.

In certain aspects, a targeted murG RNA transcript is the murG RNA transcript of a Gram-positive bacterium. In a related aspect, the Gram-positive bacterium is selected from Streptococcus, Staphylococcus, Enterococcus, and Peptostreptococcus.

In other aspects, a targeted murG RNA transcript is the murG RNA transcript of a Gram-negative bacterium. In a related aspect, the Gram negative bacterium is selected from Acinetobacter, Actinobacillus, Aeromonas, Anaplasma, Arcobacter, Avibacterium, Bacteroides, Bartonella, Bordetella, Borrelia, Brachyspira, Brucella, Campylobacter, Capnocytophaga, Chlamydia, Chlamydophila, Chryseobacterium, Coxiella, Cytophaga, Dichelobacter, Edwardsiella, Ehrlichia, Escherichia, Flavobacterium, Francisella, Fusobacterium, Gallibacterium, Haemophilus, Histophilus, Klebsiella, Lawsonia, Leptospira, Mannheimia, Megasphaera, Moraxella, Neorickettsia, Nicoletella, Ornithobacterium, Pasteurella, Photobacterium, Piscichlamydia, Piscirickettsia, Porphyromonas, Prevotella, Proteus, Pseudomonas, Rickettsia, Riemerella, Salmonella, Streptobacillus, Tenacibaculum, Vibrio, and Yersinia.

In some embodiments, the DNAzyme is linked to a cell penetration-enhancing moiety. In some embodiments, the DNAzyme is covalently linked to the cell penetration-enhancing moiety. In some embodiments, the DNAzyme is non-covalently linked to the cell penetration-enhancing moiety. In some embodiments, the DNAzyme is directly linked to the cell penetration-enhancing moiety. In some embodiments, the DNAzyme is linked to the cell penetration-enhancing moiety via a linker. In some embodiments, the cell penetration-enhancing moiety is cholesterol. In a related aspect, cholesterol is linked to the DNAzyme via an alkoxy linker. In a related aspect, cholesterol is linked to the DNAzyme via an alkyl linker.

In certain aspects, provided herein are nucleic acids comprising one or more DNAzymes described herein. In some embodiments, each DNAzyme sequence is operably linked to an origin of replication and to a termination site, and is replicated to produce one DNA DNAzyme. In some embodiments, the nucleic acid is operably linked to an origin of replication and to a termination site, and the nucleic acid comprises a cleavable sequence between each DNAzyme. In some embodiments, the cleavable sequence is a hairpin-forming sequence (e.g., an enzymatically cleavable hairpin sequence). In some embodiments, the nucleic acid is replicated and spliced to produce one or more DNA DNAzymes.

In certain aspects, provided herein are vectors comprising the nucleic acids described herein.

In certain aspects, provided herein are pharmaceutical compositions comprising the DNAzymes described herein.

In certain aspects, provided herein are pharmaceutical compositions comprising the nucleic acids or vectors described herein.

In some embodiments, the pharmaceutical compositions provided herein further comprises an antibiotic (e.g., penicillin, methicillin, cefoxitin, carbapenem, imipenem, or meropenem).

In some embodiments, the pharmaceutical compositions provided herein are for use in treating a bacterial infection. In other embodiments, the pharmaceutical compositions are for use in preventing a bacterial infection. In some embodiments, the DNAzyme in the pharmaceutical compositions is linked to a cell penetration-enhancing moiety, e.g., cholesterol. In a related aspect, the moiety is linked to the DNAzyme via an alkoxy linker.

In certain aspects, provided herein are methods of treating a bacterial infection, wherein the method comprises administering to a subject a DNAzyme described herein. In some embodiments, the DNAzyme is linked to a cell penetration-enhancing moiety, e.g., cholesterol. In a related aspect, the moiety is linked to the DNAzyme via an alkoxy linker.

In certain aspects, provided herein are methods of treating a bacterial infection, wherein the method comprises administering to a subject a vector or a nucleic acid described herein.

In certain aspects, provided herein are methods of treating a bacterial infection, wherein the method comprises administering to a subject a pharmaceutical composition described herein.

In some embodiments, the DNAzyme is linked to a cell penetration-enhancing moiety, e.g., cholesterol. In a related aspect, the moiety is linked to the DNAzyme via an alkoxy linker.

In some embodiments, the bacterial infection is caused by an antibiotic-resistant bacterium (e.g., an antibiotic-resistant Pseudomonas aeruginosa). In some embodiments, the methods provided herein further comprises administering to the subject an antibiotic (penicillin, methicillin, cefoxitin, carbapenem, imipenem, or meropenem).

In some embodiments, the subject is a mammal (e.g., a human).

In certain aspects, provided herein are methods of inhibiting bacterial growth and/or inducing death of a bacterial cell, the method comprising contacting a bacterial cell with a DNAzyme described herein. In some embodiments, the DNAzyme is linked to a cell penetration-enhancing moiety, e.g., cholesterol. In a related aspect, the moiety is linked to the DNAzyme via an alkoxy linker.

In certain aspects, provided herein are methods of inhibiting bacterial growth and/or killing a bacterial cell, the method comprising contacting a bacterial cell with a nucleic acid or a vector described herein.

In certain aspects, provided herein are methods of cleaving a murG RNA transcript, comprising contacting the murG RNA transcript with a DNAzyme described herein.

In certain aspects, provided herein are surfaces coated with a DNAzyme described herein. In some embodiments, the surface is further coated with an antibiotic (e.g., penicillin, methicillin, cefoxitin, carbapenem, imipenem, and meropenem).

### BRIEF DESCRIPTION OF FIGURES

FIG. 1A is a diagram depicting a schematic illustration of the binding of one embodiment of a DNAzyme to its target RNA. 10-23 DNAzyme is depicted, but other catalytic cores bind similarly. (B) depicts SNP trees showing conservation of murG in clinical isolates of Pseudomonas aeruginosa. The nucleotide sequence of the RNA substrate is set forth in SEQ ID NO: 42, and the nucleotide sequence of the 10-23 DNAzyme is set forth in SEQ ID NO: 43. FIGS. 1B-1F show single nucleotide polymorphism (SNP) trees based on the SNP clusters PDS000010307, PDS000010432, PDS000051135, PDS000095640, and PDS000112090. The branch length represents the SNP distance, wherein each leaf is one isolate of P. aeruginosa. The bar above each isolate shows the murG gene identity between each isolate and the ATCC BAA-3105 isolate reference.

FIGS. 2A-2D. Uptake of fluorescent DNAzyme in media mimicking in vivo conditions, in the presence of subtoxic concentrations of meropenem as judged by flow cytometry. FIG. 2A shows bacterial (Pseudomonas aeruginosa) uptake of murG-207 DNAzyme, after a 4-hr. incubation in LB medium supplemented with 32 μg/ml meropenem, as measured by qPCR. FIG. 2B is a graph showing the copy number of fluorescent murG-207 DNAzymes (Dz) per bacterium, as measured by flow cytometry. FIG. 2C is a graph showing uptake of fluorescent murG-207 DNAzyme in bacterial cells in a biofilm. Bacteria were grown under conditions favoring biofilm formation in TSB for 24 hours. Biofilm cells were separated from non-adhered cells, incubated with fluorescent murG-207 DNAzyme for 2 hours, and analyzed by flow cytometry. FIG. 2D is a graph showing uptake of DNAzymes in bacterial colonizing lung tissue samples (mouse lungs from naïve healthy mice). MDR-PA were grown with lung samples in DMEM with meropenem for 24 hours. After 24 hours, fluorescent murG-207 DNAzyme was added, and samples and surrounding media were incubated in room temperature for additional 4 hours. Cells colonizing lung tissue were extracted by moderate sonication (2-4 pulses of 5 sec, amplitude 30% per sample). In the histograms shown in FIGS. 2A, 2C, and 2D, arrows indicate which lines represent the basal fluorescence levels of the bacteria, and fluorescence of murG-207 DNAzyme with 3′cholesterol-TEG modification.

FIGS. 3A-3B. Sensitization of resistant Pseudomonas aeruginosa strain ATCC® BAA-3105™ by DNAzyme that specifically target genetic antibiotic resistance and cell wall biosynthesis (e.g. murG). FIGS. 3A-B are a heat map depicting optical density (FIG. 3A) and colony-forming assay (FIG. 3B) showing sensitization of resistant Pseudomonas aeruginosa strain ATCC® BAA-3105™ to DNAzyme that targets murG. (FIG. 3C) is a chart of MIC₉₀ levels to meropenem, as per e-test. (murG-162 is set forth in SEQ ID NO: 13; murG-207 is set forth in SEQ ID NO: 19; murG-665 is set forth in SEQ ID NO: 25; murG-366 is set forth in SEQ ID NO: 33; NT is No Treatment.)

FIGS. 4A-B are graphs showing reduction in bacterial density (FIG. 4A) or colony-forming units (FIG. 4B) (vertical axis) in biofilms grown either with or without the indicated DNAzymes (2.5 μM) in 96 wells with tryptic soy broth (TSB) for 16 hours, after which non-adhered cells were removed, and biofilms were washed and stained with 0.1% crystal violet (FIG. 4A) or replating (FIG. 4B). Average and standard mean are depicted by horizontal bars and brackets. Scr is a control scramble DNAzyme (Scr nucleotide sequence is 5′-ACCACAATACCGAGATCGGTCGTGTCGGCATGA/3CholTEG/; SEQ ID NO: 44). NT is No Treatment.

FIGS. 5A-5C: Biofilm macrostructure and morphology of single bacterial cells are affected by DNAzymes targeting murG. Biofilms were grown on top of dense stainless steel mesh in TSB in 12 well plates with or without indicated DNAzymes (1.25 μM). 24 hours afterwards, an additional dose of DNAzymes was added to treated samples. Following 48 hours of overall inoculation, biofilms were fixated and dehydrated. The mesh with associated biofilms was imaged with scanning electron microscopy (SEM). The results are of a representative field of 3 independent repeats. (FIG. 5A). NT, scr, and murG162 as above) (FIG. 5B) Enlargements of representative zones, enabling detection of single-cell morphologies. (FIG. 5C) Overall measurements of the maximal thickness of dehydrated biofilms. (NT, scr, and murG162 as above)

FIGS. 6A-6C: DNAzymes can reduce viability and increase antibiotic sensitivity of cells from pre-established biofilms. Biofilms were grown in TSB in 12 well plates. 24 hours later, the medium was replaced with PBS (FIG. 6A) or with PBS+10 μM meropenem (FIG. 6B) in the presence or absence of the indicated DNAzymes, murG-162 or SCR (scramble control), (2.5 μm). Following 8 hours, the adherent remaining cells were extracted, and the number of CFU was determined. (FIG. 6C) Biofilms were grown as described for FIG. 6B. The mesh with associated biofilms was imaged with SEM.

FIG. 7. DNAzymes can significantly reduce lung colonization and target tissue-associated infections. DNAzymes reduce viability of bacterial cells from biofilms formed in the lung tissue. MDR-PA cells were grown with mouse naïve (healthy) lung tissue samples in DMEM, and DNAzymes were added (1.25 μM) to the growth media containing the lung sample at different times post-inoculation. DNAzymes were added at the following colonization stages: “Pre-colonization”, the DNAzymes were added with the bacteria (T=0). “Mid-colonization”—the DNAzymes were added at 4 hours when a fraction of the bacteria was associated with the lung tissue. “Colonization”, the DNAzymes were added 8 hours after the inoculation of the media, where most bacteria were associated with the lung tissue. “Dispersal”, 16 hours after inoculation. For all samples, following 24 hours from bacterial inoculation, cells were harvested from the tissue and their quantity was measured by CFU analysis. (scr, murG-162, murG-207, murG-366, and murG162 as above)

FIG. 8 is a skeletal formula depicting the structure of cholesterol-TEG (triethylene glycol). The 5′ end of the cholesterol-TEG moiety may in certain embodiments be attached to the 3′ end of a DNAzyme disclosed herein.

### DETAILED DESCRIPTION

### General

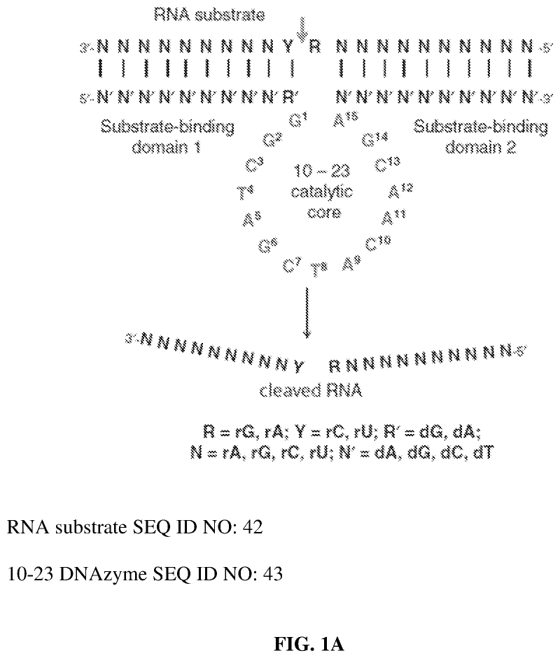

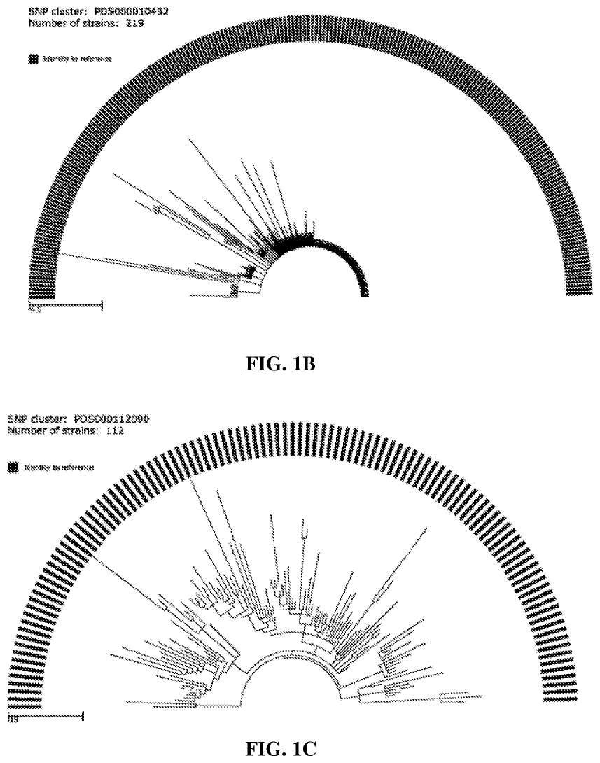

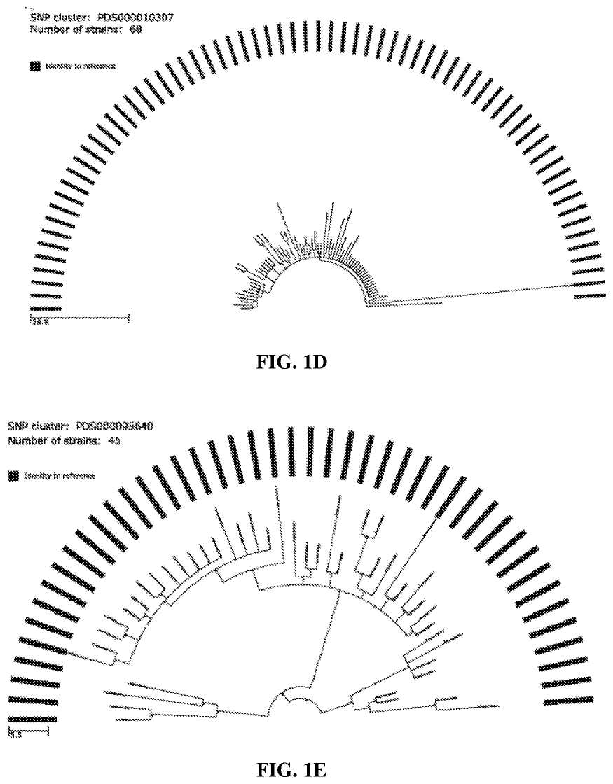

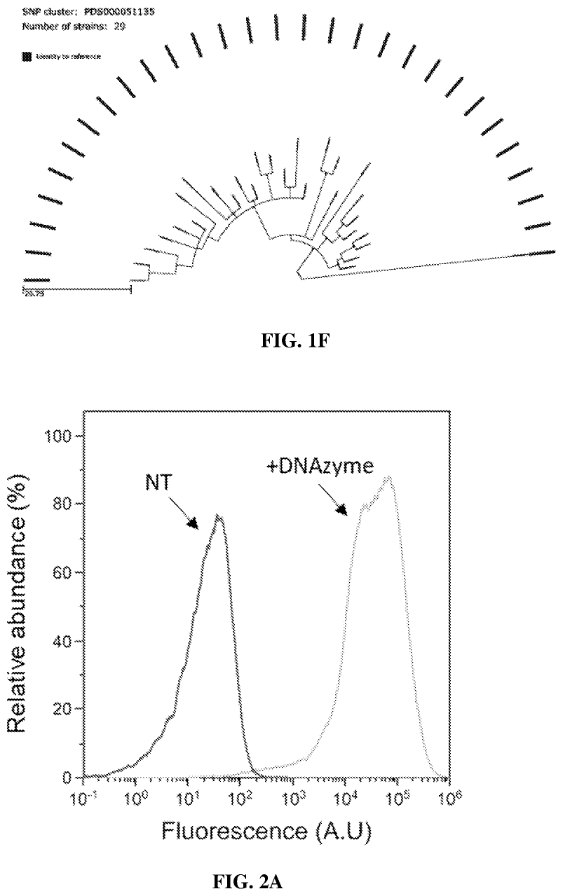

As described herein, DNAzymes are nucleic acids that bind to and cleave RNA targets. In general, a DNAzyme has a structure that includes, in 5′-3′ order: (i) a first substrate-binding domain comprising a sequence that base pairs with a first region of a RNA transcript; (ii) a DNAzyme catalytic core; and (iii) a second substrate-binding domain comprising a sequence that base pairs with a second region of the RNA transcript positioned 5′ to the first region of the RNA transcript. Upon binding of the DNAzyme to an RNA transcript, the DNAzyme catalytic core cleaves the RNA transcript at a position between the first and second region of the RNA transcript. An exemplary structure for a 10-23 type DNAzyme is illustrated in FIG. 1A. The substrate-binding domains of the DNAzyme can include DNA nucleotides, RNA nucleotides, or a combination of DNA and RNA nucleotides. There are a number of different types of DNAzyme catalytic cores known in the art, some of which are listed in Table 1.

As disclosed herein, DNAzymes targeting a murG RNA transcript are capable of cleaving and destroying murG RNA transcript, thereby inhibiting cell-wall biosynthesis and promoting antibiotic sensitivity of a bacterial cell (e.g., antibiotic-resistant Pseudomonas aeruginosa).

Therefore, in certain aspects, provided herein are DNAzymes that bind to and cleave a murG RNA transcript, inhibit cell-wall biosynthesis and/or growth of a bacterial cell, and/or induce death of a bacterial cell (e.g., antibiotic-resistant Pseudomonas aeruginosa). In certain aspects, provided herein are nucleic acids or vectors encoding such DNAzymes. In some aspects, provided herein are pharmaceutical compositions comprising such DNAzymes, nucleic acids or vectors. In some aspects, provided herein are methods of using such DNAzymes to prevent or treat bacterial infections, to inhibit cell-wall biosynthesis and/or growth of a bacterial cell, and/or to induce death of a bacterial cell.

### Definitions

For convenience, certain terms employed in the specification, examples, and appended claims are collected here.

The articles “a” and “an” are used herein to refer to one or to more than one (e.g., to at least one) of the grammatical object of the article. By way of example, “an element” means one element or more than one element.

As used herein the term “about” refers to ±10%.

As used herein, two nucleic acid sequences “complement” one another or are “complementary” to one another if they base pair one another at each position.

In certain aspects, “DNAzyme” refers to a nucleotide comprising, in 5′ to 3′ order: (i) a first substrate-binding domain comprising a sequence that base pairs with a first region of the target transcript; (ii) a DNAzyme catalytic core; and (iii) a second substrate-binding domain comprising a sequence that base pairs with a second region of the target transcript positioned 5′ to the first region of the target transcript, with a single spacing residue, typically guanine or adenine; wherein, upon binding of the DNAzyme to the target transcript, the DNAzyme catalytic core cleaves the transcript at a position between the first and second region of the transcript.

The term “DNAzyme” may include a DNAzyme of any known type, including but not limited to the types listed in Table 1. In one embodiment, the DNAzyme is a 10-23 class DNAzyme as depicted in FIG. 1A.

The terms “nucleotide base”, “nucleotide” and “nucleic acid base” are used herein interchangeably and refer to a DNA or RNA base and any modifications thereof. In certain embodiments, modified bases (which may be, for example, non-naturally occurring bases) preserving the base pair specificity of the parent DNA or RNA base are considered equivalent to the DNA or RNA parent bases, e.g., a sequence mentioned herein as containing “guanine” contain instead modified forms of guanine preserving the base pair specificity of guanine.

The terms “oligonucleotide” and “nucleic acid molecule” refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

As used herein, “specific binding” refers to the ability of a DNAzyme to bind to a predetermined target. Typically, a DNAzyme specifically binds to its target with an affinity corresponding to a K_{D} of about 10⁻⁷ M or less, about 10⁻⁸ M or less, or about 10⁻⁹ M or less and binds to the target with a K_{D} that is significantly less (e.g., at least 2 fold less, at least 5 fold less, at least 10 fold less, at least 50 fold less, at least 100 fold less, at least 500 fold less, or at least 1000 fold less) than its affinity for binding to a non-specific and unrelated target (e.g., BSA, casein, or an unrelated cell, such as an HEK 293 cell or an E. coli cell).

Throughout this application, various embodiments of DNAzymes and methods of use thereof may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DNAzymes

In certain embodiments, there is a provided a method of reducing an amount of a bacterial biofilm in a subject with a bacterial infection, comprising administering to the subject a DNAzyme targeting a transcript encoding a cell wall synthesis enzyme, thereby reducing an amount of a biofilm in the subject. In some embodiments, the bacterial strain is an antibiotic-resistant bacterial strain.

Bacteria cell wall synthesis enzymes (also referred to herein as “cell wall synthesis enzymes”) are known in the art. In certain embodiments, the targeted enzyme catalyzes one or more steps in peptidoglycan synthesis. Non-limiting examples of cell wall synthesis enzymes are MurA, MurC, MurD, MurF, MurG, MraY, FemX, FemA, FemB, and PBP2.

In some embodiments, there is a provided a method of inhibiting formation of a biofilm in a subject with a bacterial infection, comprising administering to the subject a DNAzyme targeting a transcript encoding a cell wall synthesis enzyme, thereby inhibiting formation of a biofilm in the subject. In some embodiments, the bacterial strain is an antibiotic-resistant bacterial strain.

Those skilled in the art will appreciate that, in nature and mammalian hosts, bacteria reside in structured 3D communities (biofilms), and during persistent infections, bacterial pathogens are dependent on the formation and maintenance of intact biofilms. In a biofilm, cells can be up to 1000-fold more tolerant to antibiotics due to a combination of slow growth, organic and inorganic matrices that limit diffusivity into the biofilm, and induced expression of efflux pump.

Biofilm cells are known to exhibit increased horizontal gene transfer that results in an increased competence for DNA uptake, exhibited significant intracellular uptake as well. As provided herein, intracellular DNAzyme uptake was measured in biofilm cells colonizing a lung tissue sample model, to simulate biofilms formed by P. aeruginosa in patients.

In still other embodiments, there is a provided a method of increasing antibiotic susceptibility in a subject with a bacterial infection, comprising administering to the subject a DNAzyme targeting a transcript encoding a cell wall synthesis enzyme, thereby increasing antibiotic susceptibility in a subject. In some embodiments, the bacteria are in a biofilm. Alternatively or in addition, the bacterial strain is an antibiotic-resistant bacterial strain.

In still other embodiments, there is a provided a method of enhancing antibiotic effectiveness in a subject with a bacterial infection, comprising administering to the subject a DNAzyme targeting a transcript encoding a cell wall synthesis enzyme, thereby enhancing antibiotic effectiveness in a subject. In some embodiments, the bacteria are in a biofilm. Alternatively or in addition, the bacterial strain is an antibiotic-resistant bacterial strain.

In some embodiments, the cell wall synthesis enzyme transcript is an RNA transcript of a Gram-positive bacterium (e.g., Streptococcus, Staphylococcus, including methicillin-resistant S. aureus (MRSA), Enterococcus, Gram-positive cocci, or Peptostreptococcus). In some embodiments, the Gram-positive bacteria is selected from beta-hemolytic Streptococcus, coagulase negative Staphylococcus, Enterococcus faecalis (VSE), Staphylococcus aureus, and Streptococcus pyogenes. In some embodiments, the gram-positive bacteria is selected from methicillin-sensitive Staphylococcus aureus (MSSA), and methicillin-resistant Staphylococcus aureus (MRSA), Staphylococcus aureus, Staphylococcus epidermis and other coagulase-negative staphylococci, Streptococcus pyogenes, Streptococcus pneumoniae, Streptococcus agalactiae, and Enterococcus. In some embodiments, the gram-positive bacteria are selected from Staphylococcus spp, Streptococci, Enterococcus spp, Leuconostoc spp, Corynebacterium spp, Arcanobacteria spp, Trueperella spp, Rhodococcus spp, Bacillus spp, Anaerobic Cocci, Anaerobic Gram-Positive Nonsporulating Bacilli, Actinomyces spp, Clostridium spp, Nocardia spp, Erysipelothrix spp, Listeria spp, Kytococcus spp, Mycoplasma spp, Ureaplasma spp, and Mycobacterium spp.

In some embodiments, the cell wall synthesis enzyme transcript is an RNA transcript of a Gram-negative bacterium (e.g., Acinetobacter, Actinobacillus, Aeromonas, Anaplasma, Arcobacter, Avibacterium, Bacteroides, Bartonella, Bordetella, Borrelia, Brachyspira, Brucella, Campylobacter, Capnocytophaga, Chlamydia, Chlamydophila, Chryseobacterium, Coxiella, Cytophaga, Dichelobacter, Edwardsiella, Ehrlichia, Escherichia, Flavobacterium, Francisella, Fusobacterium, Gallibacterium, Haemophilus, Histophilus, Klebsiella, Lawsonia, Leptospira, Mannheimia, Megasphaera, Moraxella, Neorickettsia, Nicoletella, Ornithobacterium, Pasteurella, Photobacterium, Piscichlamydia, Piscirickettsia, Porphyromonas, Prevotella, Proteus, Pseudomonas, Rickettsia, Riemerella, Salmonella, Streptobacillus, Tenacibaculum, Vibrio, or Yersinia). In specific embodiments, the Gram-negative bacterium is Pseudomonas aeruginosa.

In certain embodiments, the cell wall synthesis enzyme transcript is an RNA transcript of an antibiotic-resistant bacterium (e.g., antibiotic-resistant Pseudomonas aeruginosa).

In yet other embodiments, there is a provided a method of inhibiting growth of a bacterium, comprising contacting the bacteria with a DNAzyme targeting a murG RNA transcript. In some relate aspects, upon binding of the DNAzyme to the murG RNA transcript, the DNAzyme cleaves the murG RNA transcript, thereby inhibiting growth of a bacterium. As provided herein, the described DNAzymes are capable of inhibiting growth of bacterial populations. In some embodiments, the bacteria are in a biofilm. Alternatively or in addition, the bacterial strain is an antibiotic-resistant bacterial strain.

Methods for biofilm quantification are known in the art. Non-limiting embodiments of such methods, provided for exemplification, include animal models with implants were coated with strains known to form biofilms (such as Staphylococcus aureus), and biofilm amount or thickness is measured by scanning electron microscopy (SEM) and/or bioluminescent imaging. Alternatively, bacteria isolated from an implant are subjected to ex-vivo culturing on tryptic soy broth media, after which biofilm is quantified.

In other aspects, provided herein are DNAzymes targeting murG RNA transcript, as well as nucleic acids and vectors encoding such DNAzymes, and pharmaceutical compositions comprising such DNAzymes. In some aspects, provided herein are methods of using such DNAzymes, nucleic acids, vectors and/or pharmaceutical compositions for treating and/or preventing bacterial infections, inhibiting bacterial growth, reducing biofilm mass, and inducing death of bacteria.

The bacterial gene murG is an UDP-N-acetylglucosamine-N-acetylmuramyl-(pentapeptide) pyrophosphoryl-undecaprenol N-acetylglucosamine transferase that catalyzes the synthesis of the GlcNac polysaccharides of the bacterial envelope. Non-limiting examples of murG sequences are the Pseudomonas aeruginosa murG (available at the world wide web uniprot.org/uniprot/Q9HW01); and murG genes from Escherichia coli, Bacillus subtilis, Enterococcus faecium, Streptococcus pyogenes, Staphylococcus aureus, Klebsiella pneumonia, Acinetobacter baumannii, and Enterobacter cloacae, for example the sequences encoding the proteins set forth in Uniprot accession nos. P17443, P37585, AOA828QCP8, B5XMA2, A8Z3Z7, A6T4N3, BOV9F5, and V5AWE6.

Non-limiting examples of bacterial murG genes include the following:

```
(SEQ ID NO: 39; GenBank Accession No. X52644.1;
encoding E. coli murG [Uniprot accession no. P17443]);
      1 gtcgacagcc atcatgatgc tgttgcgtat tgattatgaa acgcgtctgg agaaagcgca
    61 ggcgtttgta cgaggttcac gatgagtggt caaggaaagc gattaatggt gatggcaggc
  121 ggaaccggtg gacatgtatt cccgggactg gcggttgcgc accatctaat ggctcagggt
  181 tggcaagttc gctggctggg gactgccgac cgtatggaag cggacttagt gccaaaacat
  241 ggcatcgaaa ttgatttcat tcgtatctct ggtctgcgtg gaaaaggtat aaaagcactg
  301 atagctgccc cgctgcgtat cttcaacgcc tggcgtcagg cgcgggcgat tatgaaagcg
  361 tacaaacctg acgtggtgct cggtatggga ggctacgtgt caggtccagg tggtctggcc
  421 gcgtggtcgt taggcattcc ggttgtactt catgaacaaa acggtattgc gggcttaacc
  481 aataaatggc tggcgaagat tgccaccaaa gtgatgcagg cgtttccagg tgetttccct
  541 aatgcggaag tagtgggtaa cccggtgcgt accgatgtgt tggcgctgcc gttgccgcag
  601 caacgtttgg ctggacgtga aggtccggtt cgtgtgctgg tagtgggtgg ttctcagggc
  661 gcacgcattc ttaaccagac aatgccgcag gttgctgcga aactgggtga ttcagtcact
  721 atctggcatc agagcggcaa aggttcgcaa caatccgttg aacaggcgta tgccgaagcg
  781 gggcaaccgc agcataaagt gacggaattt attgatgata tggcggcggc gtatgcgtgg
  841 gcggatgtcg tcgtttgccg ctccggtgcg ttaacggtga gtgaaatcgc cgcggcagga
  901 ctaccggcgt tgtttgtgcc gtttcaacat aaagaccgcc agcaatactg gaatgcgcta
  961 ccgctggaaa aagcgggcgc agccaaaatt atcgagcagc cacagcttag cgtggatgct
1021 gtcgccaaca ccctggccgg gtggtcgcga gaaaccttat taaccatggc agaacgcgcc
1081 cgcgctgcat ccattccgga tgccaccgag cgagtggcaa atgaagtgag ccgggttgcc
1141 cgggcgtaat tgtagcgatg ccttttgcat cgtatgaatt taagaagtta atggcgtaaa
1201 gaatgaatac acaacaattg gcaaaactgc gttccatcgt gcccgaaatg cgtcgcgttc
1261 ggcacataca ttttgtcggc attggtggtg ccggtatggg cggtattgcc gaagttctgg
1321 ccaatgaagg ttatcagatc agtggttccg atttagegcc aaatccggtc acgcagcagt
1381 taatgaatct gggtgcgacg atttatttca accatcgccc ggaaaacgta cgtgatgcca
1441 gcgtggtcgt tgtttccagc gcgatttctg ccgataaccc ggaaattgtc gccgctcatg
1501 aagcgcgtat tccggtgatc cgtcgtgccg aaatgctggc tgagttaatg cgttttcgtc
1561 atggcatcgc cattgccgga acgcacggca aaacgacaac caccgcgatg gtttccagca
1621 tctacgcaga agcggggctc gacccaacct tcgttaacgg cgggctggta aaagcggcgg
1681 gggttcatgc gcgtttgggg catggtcggt acctgattgc cgaagcagat gagagtgatg
1741 catcgttcct gcatctgcaa ccgatggtgg cgattgtcac caatatcgaa gccgaccaca
1801 tggataccta ccagggcgac tttgagaatt taaaacagac ttttattaat tttctgcaca
1861 acctgccgtt ttacggtcgt gcggtgatgt gtgttgatga tccggtgatc cgcgaattgt
1921 taccgcgagt ggggcgtcag accacgactt acggcttcag cgaagatgcc gacgtgcgtg
1981 tagaagatta tcagcagatt ggcccgcagg ggcactttac gctgctgcgc caggacaaag
2041 agccgatgeg cgtcaccctg aatgcgccag gtcgtcataa cgcgctgaac gccgcagctg
2101 cggttgcggt tgctacggaa gagggcattg acgacgaggc tattttgcgg gcgcttgaaa
2161 gcttccaggg gactggtcgc cgttttgatt tcctcggtga attcccgctg gagccagtga
2221 atggtaaaag cggtacggca atgctggtcg atgactacgg ccaccacccg acggaagtgg
2281 acgccaccat taaagcggeg cgcgcaggct ggccggataa aaacctggta atgctgtttc
2341 agccgcaccg ttttacccgt acgcgcgacc tgtatgatga tttcgccaat gtgctgacgc
2401 aggttgatac cctgttgatg ctggaagtgt atccggctgg cgaagcgcca attccgggag
2461 cggacagccg ttcgctgtgt cgcacaattc gtggacgtgg gaaaattgat cccattctgg
2521 tgccggatcc ggcgcgggta gccgagatgc tggcaccggt attaaccggt aacgacctga
2581 ttctcgttca gggggctggt aatattggaa aaattgcccg ttctttagct gaaatcaaac
2641 tgaagccgca aactccggag gaagaacaac atgactgata aaatcgcggt cctgttgggt
2701 gggacctccg ctgagcggga agtttctctg aattctggcg cagcggtgtt agccggactg
2761 cgtgaaggcg gtattgacgc gtatcctgtc gac
(SEQ ID NO: 40; GenBank Accession No. B030645.1;
encoding Streptococcus pyogenes murG);
atgcctaaga agattttatt tacaggtggt ggaactgtag gtcatgtcac cttgaacctc
attctcatac caaaatttat caaggacggt tgggaagtac attatattgg tgataaaaat
ggcattgaac atacagaaat tgaaaagtca ggccttgacg tgacctttca tgctatcgcg
acaggcaagc ttagacgcta tttttcatgg caaaatctag ctgatgtttt taaggttgca
cttggcctcc tacagtctct ctttattgtt gccaagcttc gccctcaagc ccttttttcc
aaaggtggtt ttgtctcagt accgccagtt gtggctgcta aattgcttgg taaaccagtc
tttattcatg aatcagatcg gtcaatggga ctagcaaaca agattgccta caaatttgca
actaccgtgt ataccacttt tgagcaggaa gaccagttgt ctaaagttaa acaccttgga
gcggtgacaa aggttttcaa agatgccaac caaatacctg aatcaactca gttagaggcg
gtgaaagagt attttagtag agacctaaaa accctcttgt ttattggtgg ttcggcaggg
gcgcatgtgt ttaatcagtt tattagtgat catccagaat tgaagcaacg ttataatatc
atcaatatta caggagaccc tcaccttaat gaattgagtt ctcatctgta tcgagtagat
tatgttaccg atctctacca acctttgatg gcgatggctg accttgtagt gacaagaggg
ggctctaata cactttttga gctactggca atggctaagc tacacctcat cgttcctctt
ggtaaagaag ctagccgtgg cgatcagtta gaaaatgcca cttattttga gaagaggggc
tacgctaaac aattacagga acctgattta actttgcata attttgatca ggcaatggct
gatttgtttg aacatcaggc tgattatgag gctactatgt tggcaactaa ggagattcaa
tcaccggact tcttttatga ccttttgaga gctgatatta gctccgcgat taaggagaag
taa
(SEQ ID NO: 41; GenBank Accession No. B030645.1;
encoding Staphylococcus aureus murG);
atgacgaaaa tcgcatttac cggaggggga acagttggac acgtatcagt aaatttaagt
ttaattccaa ctgcattatc acaaggatat gaagcgcttt atattggttc taaaaatggt
attgaaagag aaatgattga atcacaacta ccagaaatta agtattatcc tatttcgagt
ggtaaattaa caagatatat ttctttagaa aatgccaaaa acgtatttaa agtattgaaa
ggtattcttg atgctcgtaa agttttgaaa aaagaaaaac ctgatctatt attttcaaaa
ggtggatttg tatctgtgcc tgttgttatt gcagccaaat cattaaatat accaactatt
attcatgaat ctgacttaac accaggatta gcgaataaga tagcacttaa atttgccaag
aaaatatata caacatttga agaaacgcta aactacttac ctaaagagaa agctgatttt
attggagcaa caattcgaga agatttaaat aatggtaatg cacataatgg ttatcaatta
acaggcttta atgaaaataa aaaagtttta ctcgttatgg gtggaagctt aggaagtaaa
aaattaaata gcattattcg cgaaaactta gatgcattat tacaacaata tcaagtgata
catttaactg gtaaaggatt aaaagatgct caagttaaaa aatcaggata tatacaatat
gaatttgtta aagaggattt aacagattta ttagcaatta cggatacagt aataagtaga
gctggatcaa atgcgattta tgagttctta acattacgta taccaatgtt attagtacca
ttaggtttag atcaatcccg aggcgaccaa attgacaatg caaatcattt tgctgataaa
ggatatgcta aagcgattga tgaagaacaa ttaacagcac aaattttatt acaagaacta
aatgaaatgg tacaggaaac aactcgaatt atcaataata tgaaatcgta tgaacaaagt
tatacgaaag aagctttatt tgataagatg attaaagacg cattgaatta a
and
(SEQ ID NO: 45; ATCC BAA-3105 (E.C. 2.4.1.227; Uniprot ID Q9HW01; NCBI
Reference Sequence: NP_253102.1)
ATGAAAGGTAATGTCCTGATCATGGCGGGTGGCACCGGCGGACACGTGTTCCCGGC
GCTCGCCTGTGCGCGGGAGTTCCAGGCGCGCGGCTATGCCGTGCACTGGCTGGGGA
CGCCGCGCGGCATCGAGAATGACCTGGTACCCAAGGCCGGCCTGCCGTTGCACCTG
ATCCAGGTCAGCGGGCTGCGCGGCAAGGGCCTGAAGTCGCTGGTCAAGGCGCCGCT
GGAACTGCTCAAGTCGCTGTTCCAGGCGCTGCGGCTGATCCGCCAGCTGAGGCCGGT
CTGCGTGCTCGGCCTGGGTGGCTATGTCACCGGCCCGGGCGGCCTGGCCGCGCGCCT
CAACGGCGTGCCGCTGGTGATCCACGAGCAGAACGCCGTGGCCGGCACCGCCAACC
GCAGCCTGGCGCCGATCGCCAGGCGCGTCTGCGAGGCATTCCCGGATACCTTCCCGG
CCAGCGACAAGCGTCTGACCACCGGCAATCCGGTGCGCGCCGAGCTGTTCCTCGAC
GCGCATGCCCGCGCGCCGCTGACCGGCCGTCGGGTCAATCTGCTGGTGCTCGGCGGC
AGCCTTGGCGCGGAACCGCTGAACAAGCTGTTGCCAGAGGCCCTGGCGCAGGTGCC
GCTGGAAATCCGTCCGGCAATCCGCCATCAGGCCGGTCGTCAGCATGCCGAGATTA
CCGCCGAGCGTTATCGCACAGTGGCGGTCGAAGCGGACGTCGCGCCCTTCATCAGC
GACATGGCCGCGGCCTATGCCTGGGCCGACCTGGTGATCTGTCGCGCCGGCGCGCTG
ACCGTCAGCGAGCTGACGGCGGCGGGTTTGCCCGCCTTCCTGGTGCCGTTGCCTCAC
GCGATCGACGATCACCAGACCCGCAATGCCGAATTCCTGGTACGCAGCGGCGCCGG
CCGCCTGCTGCCGCAAAAGTCTACCGGCGCGGCCGAACTGGCCGCGCAGCTGTCCG
AGGTCCTGATGCATCCCGAAACCCTGCGCTCCATGGCAGACCAGGCACGCAGCCTG
GCGAAACCCGAGGCTACCCGGACGGTGGTCGATGCCTGCCTGGAGGTGGCCCGTGG
TTAA.
```

In some embodiments, a murG RNA transcript comprises the nucleotide sequence set forth in any of SEQ ID NO: 39, 40, 41, or 45. In some embodiments, a murG RNA transcript comprises a nucleotide sequence complementary to the nucleotide sequence set forth in any of SEQ ID NO: 39, 40, 41, or 45. In some embodiments, a murG RNA transcript comprises a portion of the nucleotide sequence set forth in any of SEQ ID NO: 39, 40, 41, or 45. In some embodiments, a murG RNA transcript comprises a nucleotide sequence complementary to a portion of the nucleotide sequence set forth in any of SEQ ID NO: 39, 40, 41, or 45.

murG was identified in an unbiased transposon screen as a factor that promotes β-lactam resistance.

In some embodiments, the DNAzyme targeting a cell wall synthesis enzyme transcript comprises, in 5′ to 3′ order: (i) a first substrate-binding domain comprising a sequence that base pairs with a first region of the transcript; (ii) a DNAzyme catalytic core; and (iii) a second substrate-binding domain comprising a sequence that base pairs with a second region of the transcript positioned 5′ to the first region of the transcript, wherein upon binding of the DNAzyme to the transcript, the DNAzyme catalytic core cleaves the transcript at a position between the first and second region of the transcript.

The DNAzyme targeting a transcript may be any type of DNAzymes, including but not limited to the types listed below in Table 1. In some embodiments, the DNAzyme catalytic core is a 10-23 catalytic core, an 8-17 catalytic core, a E1111 catalytic core, a E2112 catalytic core, a E5112 catalytic core, or a bipartite catalytic core. In some embodiments, the DNAzyme catalytic core comprises a nucleic acid sequence selected from any one of SEQ ID NOs: 1-6. In some embodiments, the DNAzyme catalytic core consists of a nucleic acid sequence selected from any one of SEQ ID NOs: 1-6.

TABLE 1

| DNAzymes | | SEQ |
|---|---|---|
| type | Catalytic Core Sequence | ID NO: |
| 10-23 | 5′ ggctagctacaacga 3′ | 1 |
| 8-17 | 5′ ccgagccggacga 3′ | 2 |
| E1111 | 5′ gtcagcgacacgaa 3′ | 3 |
| E2112 | 5′ gtcagtgactcgaa3′ | 4 |
| E5112 | 5′ gtcagctgactcgaa 4′ | 5 |
| bipartite | 5′ aggaggtaggggttccgctc 3′ | 6 |

The DNAzyme targeting an RNA transcript may bind to any region of the transcript, including 5′ untranslated region, 3′ untranslated region, or the coding region. The DNAzyme targeting an RNA transcript binds to the transcript through hybridization of the first substrate-binding domain to the first region of the transcript, and of the second substrate-binding domain to the second region of the transcript. The first substrate-binding domains can be either fully complementary to the first region of the transcript, or partially complementary to the first region of the transcript with no more than two mismatches. The second substrate-binding domains can be either fully complementary to the second region of the transcript, or partially complementary to the second region of the transcript with no more than two mismatches. Preferably, the total number of mismatches of the two substrate-binding domains to the first and second regions of the RNA transcript are no more than 3.

In some embodiments, the 5′ or 3′ arm may be 6-15 nucleotides (e.g., 6, 7, 8, 9, 10, 11, 12, 13, 14, or 15 nucleotides) in length. In some embodiments, the 5′ arm comprises 6-15 nucleotides. In some embodiments, the 5′ arm comprises 6, 7, 8, 9, 10, 11, 12, 13, 14, or 15 nucleotides. In some embodiments, the 3′ arm comprises 6-15 nucleotides. In some embodiments, the 3′ arm comprises 6, 7, 8, 9, 10, 11, 12, 13, 14, or 15 nucleotides. In other embodiments, the 3′ and 5′ arms are the same or different lengths. In some embodiments, the 5′ arm and the 3′ arm are each 6, 7, 8, 9, 10, 11, 12, 13, 14, or 15 nucleotides in length. In some embodiments, the 5′ arm and the 3′ arm are each 9 nucleotides in length. In some embodiments, the 5′ arm and the 3′ arm comprise different lengths of nucleotides independently selected from 6, 7, 8, 9, 10, 11, 12, 13, 14, or 15 nucleotides. These embodiments may be freely combined with each other.

In some embodiments, the first substrate-binding domain comprises a nucleic acid sequence 5′-GATCAGGTG-3′ (SEQ ID NO: 7) and the second substrate-binding domain comprises a nucleic acid sequence 5′-AACGGCAGG-3′ (SEQ ID NO: 8). In some embodiments, the first substrate-binding domain comprises a nucleic acid sequence 5′-CTTGACCAG-3′ (SEQ ID NO: 9) and the second substrate-binding domain comprises a nucleic acid sequence 5′-GACTTCAGG-3′ (SEQ ID NO: 10). In some embodiments, the first substrate-binding domain comprises a nucleic acid sequence 5′-ATCTCGGCA-3′ (SEQ ID NO: 11) and the second substrate-binding domain comprises a nucleic acid sequence 5′-GCTGACGAC-3′ (SEQ ID NO: 12). In some embodiments, the nucleic acid sequence of the first substrate-binding domain comprises 5′-GGCGTTCTG-3′ (SEQ ID NO: 31), and sequence of the second substrate-binding domain comprises 5′-TCGTGGATC-3′ (SEQ ID NO: 32).

In some embodiments, the first substrate-binding domain consists of the nucleic acid sequence 5′-GATCAGGTG-3′ (SEQ ID NO: 7) and the second substrate-binding domain consists of the nucleic acid sequence 5′-AACGGCAGG-3′ (SEQ ID NO: 8). In some embodiments, the first substrate-binding domain consists of the nucleic acid sequence 5′-CTTGACCAG-3′ (SEQ ID NO: 9) and the second substrate-binding domain consists of the nucleic acid sequence 5′-GACTTCAGG-3′ (SEQ ID NO: 10). In some embodiments, the first substrate-binding domain consists of the nucleic acid sequence 5′-ATCTCGGCA-3′ (SEQ ID NO: 11) and the second substrate-binding domain consists of the nucleic acid sequence 5′-GCTGACGAC-3′ (SEQ ID NO: 12). In some embodiments, the nucleic acid sequence of the first substrate-binding domain consists of the nucleic acid sequence 5′-GGCGTTCTG-3′ (SEQ ID NO: 31), and sequence of the second substrate-binding domain consists of the nucleic acid sequence 5′-TCGTGGATC-3′ (SEQ ID NO: 32).

In some embodiments, the first and second substrate-binding domains of a DNAzyme used in a method disclosed here for reducing an amount of a biofilm in a subject with a bacterial infection comprises the nucleotide sequences set forth in SEQ ID NO: 7 and SEQ ID NO: 8, respectfully. In some embodiments, the first and second substrate-binding domains of a DNAzyme used in a method disclosed here for reducing an amount of a biofilm in a subject with a bacterial infection comprises the nucleotide sequences set forth in SEQ ID NO: 9 and SEQ ID NO: 10, respectfully. In some embodiments, the first and second substrate-binding domains of a DNAzyme used in a method disclosed here for reducing an amount of a biofilm in a subject with a bacterial infection comprises the nucleotide sequences set forth in SEQ ID NO: 11 and SEQ ID NO: 12, respectfully. In some embodiments, the first and second substrate-binding domains of a DNAzyme used in a method disclosed here for reducing an amount of a biofilm in a subject with a bacterial infection comprises the nucleotide sequences set forth in SEQ ID NO: 31 and SEQ ID NO: 32, respectfully.

In some embodiments, the first and second substrate-binding domains of a DNAzyme used in a method disclosed here for increasing antibiotic susceptibility in a subject with a bacterial infection comprises the nucleotide sequences set forth in SEQ ID NO: 7 and SEQ ID NO: 8, respectfully. In some embodiments, the first and second substrate-binding domains of a DNAzyme used in a method disclosed here for increasing antibiotic susceptibility in a subject with a bacterial infection comprises the nucleotide sequences set forth in SEQ ID NO: 9 and SEQ ID NO: 10, respectfully. In some embodiments, the first and second substrate-binding domains of a DNAzyme used in a method disclosed here for increasing antibiotic susceptibility in a subject with a bacterial infection comprises the nucleotide sequences set forth in SEQ ID NO: 11 and SEQ ID NO: 12, respectfully. In some embodiments, the first and second substrate-binding domains of a DNAzyme used in a method disclosed here for increasing antibiotic susceptibility in a subject with a bacterial infection comprises the nucleotide sequences set forth in SEQ ID NO: 31 and SEQ ID NO: 32, respectfully.

In some embodiments, the first and second substrate-binding domains of a DNAzyme used in a method disclosed here for inhibiting bacterial growth in a subject with a bacterial infection comprises the nucleotide sequences set forth in SEQ ID NO: 7 and SEQ ID NO: 8, respectfully.

In some embodiments, the first and second substrate-binding domains of a DNAzyme used in a method disclosed here for inhibiting bacterial growth in a subject with a bacterial infection comprises the nucleotide sequences set forth in SEQ ID NO: 9 and SEQ ID NO: 10, respectfully.

In some embodiments, the first and second substrate-binding domains of a DNAzyme used in a method disclosed here for inhibiting bacterial growth in a subject with a bacterial infection comprises the nucleotide sequences set forth in SEQ ID NO: 11 and SEQ ID NO: 12, respectfully.

In some embodiments, the first and second substrate-binding domains of a DNAzyme used in a method disclosed here for inhibiting bacterial growth in a subject with a bacterial infection comprises the nucleotide sequences set forth in SEQ ID NO: 31 and SEQ ID NO: 32, respectfully.

In all substrate-binding domain sequences disclosed herein where a thymidine base is identified, a uracil base is also contemplated.

The pairs of the substrate-binding domains described above can be combined with any catalytic core sequence described herein to form DNAzymes that targets the murG RNA transcript. Such murG-targeting DNAzymes may comprise a nucleic acid sequence selected from SEQ ID NOs: 13-38 in Table 2 below.

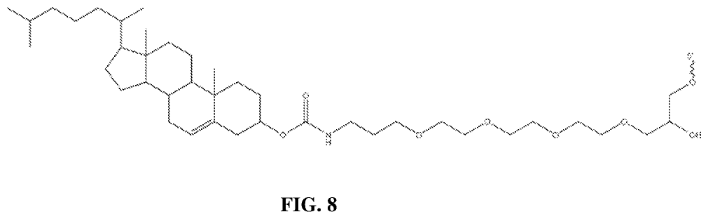

TABLE 2
DNAzymes targeting a murG RNA transcript. Chol-TEG-3′ refers to a
cholesterol-TEG modification attached to the 3′ end of the
DNAzyme nucleotide sequence. (FIG. 8 )

| DNAzyme Sequences | SEQ ID NO: |
|---|---|
| 5′-GATCAGGTG ggctagctacaacga AACGGCAGG-Chol-TEG-3′ | 13 |
| 5′-GATCAGGTG ccgagccggacga AACGGCAGG-Chol-TEG-3′ | 14 |
| 5′-GATCAGGTG gtcagcgacacgaa AACGGCAGG-Chol-TEG-3′ | 15 |
| 5′-GATCAGGTG gtcagtgactcgaa AACGGCAGG-Chol-TEG-3′ | 16 |
| 5′-GATCAGGTG gtcagctgactcgaa AACGGCAGG-Chol-TEG-3′ | 17 |
| 5′-GATCAGGTG aggaggtaggggttccgctc AACGGCAGG-Chol-TEG-3′ | 18 |
| 5′-CTTGACCAG ggctagctacaacga GACTTCAGG-Chol-TEG-3′ | 19 |
| 5′-CTTGACCAG ccgagccggacga GACTTCAGG-Chol-TEG-3′ | 20 |
| 5′-CTTGACCAG gtcagcgacacgaa GACTTCAGG-Chol-TEG-3′ | 21 |
| 5′-CTTGACCAG gtcagtgactcgaa GACTTCAGG-Chol-TEG-3′ | 22 |
| 5′-CTTGACCAG gtcagctgactcgaa GACTTCAGG-Chol-TEG-3′ | 23 |
| 5′-CTTGACCAG aggaggtaggggttccgctc GACTTCAGG-Chol-TEG-3′ | 24 |
| 5′-ATCTCGGCA ggctagctacaacga GCTGACGAC-Chol-TEG-3′ | 25 |
| 5′-ATCTCGGCA ccgagccggacga GCTGACGAC-Chol-TEG-3′ | 26 |
| 5′-ATCTCGGCA gtcagcgacacgaa GCTGACGAC-Chol-TEG-3′ | 27 |
| 5′-ATCTCGGCA gtcagtgactcgaa GCTGACGAC-Chol-TEG-3′ | 28 |
| 5′-ATCTCGGCA gtcagctgactegaa GCTGACGAC-Chol-TEG-3′ | 29 |
| 5′-ATCTCGGCA aggaggtaggggttccgctc GCTGACGAC-Chol-TEG-3′ | 30 |
| 5′-GGCGTTCTG ggctagctacaacga TCGTGGATC-Chol-TEG-3′ | 33 |
| 5′-GGCGTTCTG ccgagccggacga TCGTGGATC-Chol-TEG-3′ | 34 |
| 5′-GGCGTTCTG gtcagcgacacgaa TCGTGGATC-Chol-TEG-3′ | 35 |
| 5′-GGCGTTCTG gtcagtgactegaa TCGTGGATC-Chol-TEG-3′ | 36 |
| 5′-GGCGTTCTG gtcagctgactegaa TCGTGGATC-Chol-TEG-3′ | 37 |
| 5′-GGCGTTCTG aggaggtaggggttccgctc TCGTGGATC-Chol-TEG-3′ | 38 |

In some embodiments, a DNAzyme used in a method disclosed here for reducing an amount of a biofilm in a subject with a bacterial infection comprises the nucleotide sequence set forth in any of SEQ ID NO: 13-38. In some embodiments, a DNAzyme used in a method disclosed here for increasing antibiotic susceptibility in a subject with a bacterial infection comprises the nucleotide sequence set forth in any of SEQ ID NO: 13-38. In some embodiments, a DNAzyme used in a method disclosed here for inhibiting bacterial growth in a subject with a bacterial infection comprises the nucleotide sequence set forth in any of SEQ ID NO: 13-38.

In specific embodiments, the DNAzymes targeting a murG RNA transcript from a Pseudomonas aeruginosa cell are shown below in Table 3.

TABLE 3
Exemplary DNAzymes effective against Pseudomonas aeruginosa
cell wall biosynthesis and antibiotic production.

| | Cleavage | |
|---|---|---|
| | site in | |
| | SEQ ID | |
| Target | NO: 45, | |
| gene | 5′ to nt # | DNAzyme sequence |
| murG | 162 | GATCAGGTGggctagctacaacgaAACGGCAGG/3CholTEG |
| | 207 | CTTGACCAGggctagctacaacgaGACTTCAGG/3CholTEG |
| | 665 | ATCTCGGCAggctagctacaacgaGCTGACGAC/3CholTEG |
| | 366 | GGCGTTCTGggctagctacaacgaTCGTGGATC/3CholTEG |

In some embodiments, the DNAzymes provided herein are able to cleave a murG RNA transcript. In some embodiments, the DNAzymes provided herein are able to reduce expression level (e.g., RNA transcript level and/or protein level) of murG. In some embodiments, the DNAzymes are able to inhibit bacterial cell wall biosynthesis. In some embodiments, the DNAzymes are able to inhibit bacterial cell growth and/or to kill a bacterial cell in vitro. In some embodiments, the DNAzymes are able to inhibit bacterial cell growth and/or to induce death of a bacterial cell in vivo. In some embodiments, the DNAzymes are able to prevent, treat, or impede progression of a bacterial infection.

In some embodiments, the murG RNA transcript is the murG RNA transcript of a Gram-positive bacterium (e.g., Streptococcus, Staphylococcus, including methicillin-resistant S. aureus (MRSA), Enterococcus, Gram-positive cocci, or Peptostreptococcus). In some embodiments, the Gram-positive bacteria is selected from beta-hemolytic Streptococcus, coagulase negative Staphylococcus, Enterococcus faecalis (VSE), Staphylococcus aureus, and Streptococcus pyogenes. In some embodiments, the gram-positive bacteria is selected from methicillin-sensitive Staphylococcus aureus (MSSA), and methicillin-resistant Staphylococcus aureus (MRSA), Staphylococcus aureus, Staphylococcus epidermis and other coagulase-negative staphylococci, Streptococcus pyogenes, Streptococcus pneumoniae, Streptococcus agalactiae, and Enterococcus. In some embodiments, the gram-positive bacteria are selected from Staphylococcus spp, Streptococci, Enterococcus spp, Leuconostoc spp, Corynebacterium spp, Arcanobacteria spp, Trueperella spp, Rhodococcus spp, Bacillus spp, Anaerobic Cocci, Anaerobic Gram-Positive Nonsporulating Bacilli, Actinomyces spp, Clostridium spp, Nocardia spp, Erysipelothrix spp, Listeria spp, Kytococcus spp, Mycoplasma spp, Ureaplasma spp, and Mycobacterium spp. In some embodiments, the murG RNA transcript is the murG RNA transcript of a Gram-negative bacterium (e.g., Acinetobacter, Actinobacillus, Aeromonas, Anaplasma, Arcobacter, Avibacterium, Bacteroides, Bartonella, Bordetella, Borrelia, Brachyspira, Brucella, Campylobacter, Capnocytophaga, Chlamydia, Chlamydophila, Chryseobacterium, Coxiella, Cytophaga, Dichelobacter, Edwardsiella, Ehrlichia, Escherichia, Flavobacterium, Francisella, Fusobacterium, Gallibacterium, Haemophilus, Histophilus, Klebsiella, Lawsonia, Leptospira, Mannheimia, Megasphaera, Moraxella, Neorickettsia, Nicoletella, Ornithobacterium, Pasteurella, Photobacterium, Piscichlamydia, Piscirickettsia, Porphyromonas, Prevotella, Proteus, Pseudomonas, Rickettsia, Riemerella, Salmonella, Streptobacillus, Tenacibaculum, Vibrio or Yersinia). In specific embodiments, the Gram-negative bacterium is Pseudomonas aeruginosa.

In certain embodiments, the murG RNA transcript is the murG RNA transcript of an antibiotic-resistant bacterium (e.g., antibiotic-resistant Pseudomonas aeruginosa).

In some embodiments, provided herein are methods of inhibiting expression of a gene, comprising contacting the RNA transcript with a DNAzyme described herein. In some embodiments, where an intact cell is the target, a cell containing the RNA transcript is contacted with the DNAzyme. In further embodiments, the DNAzyme is conjugated to a moiety that enables cell penetration. In still other embodiments, an RNA transcript is contacted with the described DNAzyme in vitro. In some embodiments, a method of inhibiting expression of a gene comprises reducing the mRNA level of the gene, reducing the level of the encoded protein, or reducing the levels of both mRNA and it encoded protein. In some embodiments, methods of inhibiting expression of a gene result in cytotoxicity of the cell comprising said gene.

In some embodiments, the DNAzymes provided herein are able to inhibit bacterial cell growth and/or to induce death of a bacterial cell in combination with an antibiotic (e.g., penicillin, methicillin, cefoxitin, carbapenem, imipenem, or meropenem). In some embodiments, the administration of the DNAzyme is in combination with the antibiotic

In some embodiments, the DNAzymes provided herein comprise one or more chemical modifications. In some embodiments, the one or more chemical modifications are selected from the group consisting of base modifications, sugar modifications and internucleotide linkage modifications. In some embodiments, the one or more chemical modifications are selected are selected from the group consisting of locked nucleic acids (LNA), phosphorothioate, 2-O-fluoro, 2-O-methyl, 2-O-methoxyethyl, and methyl-Cytosine.

Some embodiments of modifications are provided in Table 4.

TABLE 4
Exemplary chemical modifications.

| | Sugar | | |
|---|---|---|---|
| Terminal | ring | Nitrogen base | Backbone |
| biotin | 2′-OH | BzdU | Phosphorothioate |
| | (RNA) | | |
| Inverted-dT | 2′- | Naphtyl | Methylphosphorothioate |
| | OMe | | |
| PEG (0.5-40 kDa) | 2′-F | Triptamino | Phosphorodithioate |
| Cholesterol | 2′- | Isobutyl | Triazole |
| | NH2 | | |
| Albumin | LNA | 5-Methyl Cytosine | Amide (PNA) |
| Chitin (0.5-40 kDa) | UNA | Alkyne | Alkyne |
| | | (dibenzocyclooctyne) | (dibenzocyclooctyne) |
| Chitosan (0.5- | 2′-F | Azide | Azide |
| 40 kDa) | ANA | | |
| Cellulose (0.5- | L- | Maleimide | Maleimide |
| 40 kDa) | DNA | | |
| Terminal amine | CeNA | | |
| (alkyne chain with | | | |
| amine) | | | |
| Alkyl | TNA | | |
| (dibenzocyclooctyne) | | | |
| Azide | HNA | | |
| Thiol | | | |
| Maleimide | | | |
| NHS | | | |

In some embodiments, the DNAzyme comprises deoxyribonucleotides. In other embodiments, the DNAzyme comprises ribonucleotides. In still other embodiments, the DNAzyme comprises a combination of deoxyribonucleotides and ribonucleotides.

In certain embodiments, the DNAzymes comprise a terminal modification. In some embodiments, the DNAzymes are chemically modified with poly-ethylene glycol (PEG) (e.g., 0.5-40 kDa) (e.g., attached to the 5′ end of the DNAzyme). In some embodiments, the DNAzymes comprise a 5′ end cap (e.g., is an inverted thymidine, biotin, albumin, chitin, chitosan, cellulose, terminal amine, alkyne, azide, thiol, maleimide, NHS). In certain embodiments, the DNAzymes comprise a 3′ end cap (e.g., is an inverted thymidine, biotin, albumin, chitin, chitosan, cellulose, terminal amine, alkyne, azide, thiol, maleimide, NHS).

In certain embodiments, the DNAzymes provided herein comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, or 50) modified sugars. In some embodiments, the DNAzymes comprise one or more 2′ sugar substitutions (e.g., a 2′-fluoro, a 2′-amino, or a 2′-O-methyl substitution). In certain embodiments, the DNAzymes comprise locked nucleic acid (LNA), unlocked nucleic acid (UNA) and/or 2′deozy-2′fluoro-D-arabinonucleic acid (2′-F ANA) sugars in their backbone.

In certain embodiments, the DNAzymes comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 3031, 32, 33, 34, 35, 36, 37, 38, 39, 40, 41, 42, 43, 44, 45, 46, 47, 48, 49, or 50) methylphosphonate internucleotide bonds and/or a phosphorothioate (PS) internucleotide bonds. In certain embodiments, the DNAzymes comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, or 50) triazole internucleotide bonds. In certain embodiments, the DNAzymes are modified with a cholesterol or a dialkyl lipid (e.g., on their 5′ end).

In some embodiments, the guanine-rich DNAzymes comprise one or more modified bases (e.g., 5-(N-benzylcarboxyamide)-2′-deoxyuridine) [5-BzdU], Q-naphthyl-, tryptamine, or Isobutyl substituted bases; 5-methyl cytosine, or bases modified with alkyne, dibenzocyclooctyne, azide, or maleimide).

In certain embodiments, the DNAzymes provided herein are DNA DNAzymes (e.g., D-DNA DNAzymes or enantiomer L-DNA DNAzymes). In some embodiments, the DNAzymes provided herein are RNA DNAzymes (e.g., D-RNA DNAzymes or enantiomer L-RNA DNAzymes). In some embodiments, the DNAzymes comprise a mixture of DNA and RNA.

In certain aspects, the DNAzymes provided herein are linked to a penetration-enhancing moiety. The cell penetration-enhancing moiety may be, e.g., an aptamer, a small molecule, a polypeptide, a nucleic acid, a protein, or an antibody. In some embodiments, the DNAzyme is covalently linked to the penetration-enhancing moiety. In some embodiments, the DNAzyme is non-covalently linked to the penetration-enhancing moiety. In some embodiments, the DNAzyme is directly linked to the penetration-enhancing moiety. In some embodiments, the DNAzyme is linked to the penetration-enhancing moiety via a linker.

The term “penetration-enhancing moiety” refers to any moiety known in the art to facilitate actively or passively or enhance penetration of the compound into the cells. In some embodiments, the penetration-enhancing moiety is a polysaccharide, synthetic nucleoside base, inverted nucleoside base, cholesterol, other sterols, lipids, membrane lipids, or synthetic lipids. In certain embodiments, the penetration-enhancing moiety is a moiety that enhances the permeability of a target cell.

In some embodiments, the cell penetration-enhancing moiety is cholesterol, linked directly or via a linker to the 5′ or 3′ terminus (or both) of the DNAzyme. In certain embodiments, the linker is an alkyl or alkoxy group, a non-limiting examples of which is cholesterol-TEG (triethylene glycol), whose structure is depicted in FIG. 8. In some embodiments, the chain length of the linker is between 5-20, in other embodiments between 5-16, in other embodiments between 10-16 atoms.

In some embodiments, the DNAzyme is encapsulated in a liposome, conjugated to a micro- or nano-particle, or embedded in a polymer matrix such as gel, PLGA, PEG, etc.

DNAzymes may be synthesized by methods which are well known to the skilled person. For example, DNAzymes may be chemically synthesized, e.g. on a solid support. Solid phase synthesis may use phosphoramidite chemistry. Briefly, the synthesis cycle starts with the removal of the acid-labile 5′-dimethoxytrityl protection group (DMT, “Trityl”) from the hydroxyl function of the terminal, support-bound nucleoside by UV-controlled treatment with an organic acid. The exposed highly-reactive hydroxyl group is then available to react in the coupling step with the next protected nucleoside phosphoramidite building block, forming a phosphite triester backbone. Next, the acid-labile phosphite triester backbone is oxidized to the stable pentavalent phosphate trimester. If a phosphorothioate modification is desired at a specific backbone position, the acid labile phosphite trimester backbone is sulfuridized at this step, instead of the oxidation process, to generate a P═S bond rather than a P═O. Successively, all the unreacted 5′-hydroxyl groups are acetylated (“capped”) in order to block these sites during the next coupling step, avoiding internal mismatch sequences. Following the capping step, the cycle starts again by removal of the DMT-protection group and successive coupling of the next base according to the desired sequence.

Finally, the oligonucleotide is cleaved from the solid support, and all protection groups are removed from the backbone and bases.

### Nucleic Acids/Vectors

In certain aspects, the present invention provides a nucleic acid comprising one or more DNAzymes described herein. In some embodiments, each DNAzyme sequence is operably linked to an origin of replication and to a termination site.

The term “operably linked” as used herein refers to an arrangement of elements that allows them to be functionally related.

In some embodiments, each DNAzyme sequence is operably linked to an origin of replication and to a termination site such that each DNAzyme is separately replicated by the bacterial DNA replication machinery.

In other embodiments, the whole nucleic acid comprising one or more DNAzymes is operably linked to an origin of replication and to a termination site, wherein the nucleic acid comprises a cleavable sequence between each DNAzyme sequence. The cleavable sequence may be a hairpin-forming sequence, e.g., an enzymatically cleavable hairpin. In some embodiments, the nucleic acid comprising one or more DNAzymes is replicated by the bacterial DNA replication machinery and consequently spliced or parsed either via self-splicing or via enzymatic splicing to produce one or more DNA DNAzymes.

In certain aspects, there is provided a nucleic acid comprising complementary sequences of one or more DNAzymes described herein.

In some embodiments, the complementary sequence of each DNAzyme is operably linked to a promoter. In some embodiments, the complementary sequence of each DNAzyme is transcribed to produce a RNA DNAzyme.

In other embodiments, the whole nucleic acid comprising complementary sequences of one or more DNAzymes is operably linked to a promoter, wherein the nucleic acid encodes a cleavable sequence between the complementary sequence of each DNAzyme. In some embodiments, the cleavable sequence is a hairpin-forming sequence, e.g., an enzymatically cleavable hairpin. In some embodiments, the nucleic acid comprising complementary sequences of one or more DNAzymes is transcribed by the bacterial transcription machinery and consequently spliced or parsed either via self-splicing or via enzymatic splicing to obtain one or more RNA DNAzymes.

In certain aspects, disclosed herein is a vector comprising a nucleic acid described herein.

The terms “vector” and “expression vector” are used herein interchangeably, having all the same meanings and qualities, and may encompass any viral or non-viral vector such as plasmid, virus, retrovirus, bacteriophage, cosmid, artificial chromosome (bacterial or yeast), phage, binary vector in double or single stranded linear or circular form, or nucleic acid sequence which is able to transform host cells and optionally capable of replicating in a host cell. The vector may contain an optional marker suitable for use in the identification of transformed cells, e.g., tetracycline resistance or ampicillin resistance. A cloning vector may or may not possess the features necessary for it to operate as an expression vector.

In one embodiment, the vector is a plasmid. In another embodiment, the vector is a phage.

In some embodiments, DNAzyme oligonucleotides are obtained upon replication or transcription of a vector described herein.

In some embodiments, the vector described herein is conjugated to a penetration-enhancing moiety.

### Pharmaceutical Compositions

In certain aspects, provided herein are pharmaceutical compositions comprising a DNAzyme (e.g., a therapeutically effective amount of a DNAzyme) provided herein. In certain aspects, provided herein are pharmaceutical compositions comprising a nucleic acid or a vector that comprises or encodes a DNAzyme (e.g., a therapeutically effective amount of a nucleic acid or a vector). In some embodiments, the pharmaceutical compositions provided herein further comprise an antibiotic (e.g., penicillin, methicillin, cefoxitin, carbapenem, imipenem, and meropenem). In some embodiments, the pharmaceutical compositions provided herein further comprise a pharmaceutically acceptable carrier.

In some embodiments, the pharmaceutical composition comprises a plurality (e.g., 2, 3, 4, 5, 6, 7, 8, 9, 10, 20, or more) of DNAzymes described herein. In some embodiments, the pharmaceutical composition comprises a plurality of DNAzymes are at equal percentage. In some embodiments, the pharmaceutical composition comprises a plurality of DNAzymes in varying ratios.

In some embodiments, the bacterial infection is caused by a Gram-positive bacterium (e.g., Streptococcus, Staphylococcus including methicillin-resistant S. aureus (MRSA), Enterococcus, Gram-positive cocci, or Peptostreptococcus). In some embodiments, the Gram-positive bacteria is selected from beta-hemolytic Streptococcus, coagulase negative Staphylococcus, Enterococcus faecalis (VSE), Staphylococcus aureus, and Streptococcus pyogenes. In some embodiments, the gram-positive bacteria is selected from methicillin-sensitive Staphylococcus aureus (MSSA), and methicillin-resistant Staphylococcus aureus (MRSA), Staphylococcus aureus, Staphylococcus epidermis and other coagulase-negative staphylococci, Streptococcus pyogenes, Streptococcus pneumoniae, Streptococcus agalactiae, and Enterococcus. In some embodiments, the gram-positive bacteria are selected from Staphylococcus spp, Streptococci, Enterococcus spp, Leuconostoc spp, Corynebacterium spp, Arcanobacteria spp, Trueperella spp, Rhodococcus spp, Bacillus spp, Anaerobic Cocci, Anaerobic Gram-Positive Nonsporulating Bacilli, Actinomyces spp, Clostridium spp, Nocardia spp, Erysipelothrix spp, Listeria spp, Kytococcus spp, Mycoplasma spp, Ureaplasma spp, and Mycobacterium spp.

In some embodiments, the bacterial infection is caused by a Gram-negative bacterium (e.g., Acinetobacter, Actinobacillus, Aeromonas, Anaplasma, Arcobacter, Avibacterium, Bacteroides, Bartonella, Bordetella, Borrelia, Brachyspira, Brucella, Campylobacter, Capnocytophaga, Chlamydia, Chlamydophila, Chryseobacterium, Coxiella, Cytophaga, Dichelobacter, Edwardsiella, Ehrlichia, Escherichia, Flavobacterium, Francisella, Fusobacterium, Gallibacterium, Haemophilus, Histophilus, Klebsiella, Lawsonia, Leptospira, Mannheimia, Megasphaera, Moraxella, Neorickettsia, Nicoletella, Ornithobacterium, Pasteurella, Photobacterium, Piscichlamydia, Piscirickettsia, Porphyromonas, Prevotella, Proteus, Pseudomonas, Rickettsia, Riemerella, Salmonella, Streptobacillus, Tenacibaculum, Vibrio or Yersinia). In specific embodiments, the Gram-negative bacterium is Pseudomonas aeruginosa. In certain embodiments, the bacterial cell is antibiotic-resistant (e.g., antibiotic-resistant Pseudomonas aeruginosa).

Formulation of the pharmaceutical composition may be adjusted according to applications. In particular, the pharmaceutical composition may be formulated using a method known in the art to provide rapid, continuous or delayed release of the active ingredient after administration to mammals.

In some embodiments, the pharmaceutical composition is in a form selected from the group consisting of tablets, pills, capsules, pellets, granules, powders, lozenges, sachets, cachets, elixirs, suspensions, dispersions, emulsions, solutions, infusions, syrups, aerosols, ophthalmic ointments, ointments, soft and hard gelatin capsules, suppositories, sterile injectable solutions, and sterile packaged powders.

In some embodiments, the pharmaceutical composition is suitable for administration via a route selected from the group consisting of oral, rectal, intramuscular, subcutaneous, intravenous, intraperitoneal, inhaled, intranasal, intraarterial, intravesicle, intraocular, transdermal and topical.

The composition for oral administration may be in a form of tablets, troches, lozenges, aqueous, or oily suspensions, dispersible powders or granules, emulsions, hard or soft capsules, or syrups or elixirs. Pharmaceutical compositions intended for oral use may be prepared according to any method known to the art for the manufacture of pharmaceutical compositions and may further comprise one or more agents selected from sweetening agents, flavoring agents, coloring agents and preserving agents in order to provide pharmaceutically elegant and palatable preparations. Tablets contain the active agent in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binders; and lubricating agents. The tablets are preferably coated utilizing known techniques to delay disintegration and absorption in the gastrointestinal tract and thereby provide an extended release of the drug over a longer period.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions. Solid carriers or excipients are, for example, lactose, starch or talcum or liquid carriers such as, for example, water, fatty oils or liquid paraffins.

Other carriers or excipients which may be used include, but are not limited to, materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminium silicates; and amino acids having from 2 to 12 carbon atoms and derivatives thereof such as, but not limited to, glycine, L-alanine, L-aspartic acid, L-glutamic acid, L-hydroxyproline, L-isoleucine, L-leucine and L-phenylalanine. Each possibility represents a separate embodiment of the present invention.

Solutions or suspensions used for parenteral, intradermal, or subcutaneous application typically include the following components: a sterile diluent such as water for injection, saline solution, fixed oils, polyethylene glycols, glycerine, propylene glycol (or other synthetic solvents), antibacterial agents (e.g., benzyl alcohol, methyl parabens), antioxidants (e.g., ascorbic acid, sodium bisulfite), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials.

Pharmaceutical compositions adapted for parenteral administration include, but are not limited to, aqueous and non-aqueous sterile injectable solutions or suspensions, which can contain antioxidants, buffers, bacteriostats and solutes that render the compositions substantially isotonic with the blood of an intended recipient. Such compositions can also comprise water, alcohols, polyols, glycerine and vegetable oils, for example. Extemporaneous injection solutions and suspensions can be prepared from sterile powders, granules and tablets. Such compositions preferably comprise a therapeutically effective amount of a compound of the invention and/or other therapeutic agent(s), together with a suitable amount of carrier to provide the form for proper administration to the subject.

The terms “pharmaceutically acceptable” and “pharmacologically acceptable” include molecular entities and compositions that do not produce an adverse, allergic, or other untoward reactions when administered to an animal, or human, as appropriate. For human administration, preparations should meet sterility, pyrogenicity, general safety, and purity standards as required by a government drug regulatory agency, e.g., the United States Food and Drug Administration (FDA) Office of Biologics standards.

In some embodiments, the pharmaceutical composition is formulated to enhance the penetration of DNAzymes, nucleic acids, or vectors described herein into bacterial cells.

### Therapeutic Methods and Other Uses

In some aspects, provided herein are methods of treating a bacterial infection, comprising administering to a subject one or more DNAzymes, one or more nucleic acids, or one or more vectors described herein.

In other aspects, provided herein are methods of treating a bacterial infection, comprising administering to a subject a pharmaceutical composition provided herein.

In other aspects, provided herein are methods of preventing worsening of a bacterial infection, comprising administering to a subject a pharmaceutical composition provided herein.

In other aspects, provided herein are methods of inhibiting progress of a bacterial infection, comprising administering to a subject a pharmaceutical composition provided herein.

In some embodiments, the bacterial infection is caused by a Gram-positive bacterium (e.g., Streptococcus, Staphylococcus including methicillin-resistant S. aureus (MRSA), Enterococcus, Gram-positive cocci, or Peptostreptococcus). In some embodiments, the Gram-positive bacterium is selected from beta-hemolytic Streptococcus, coagulase negative Staphylococcus, Enterococcus faecalis (VSE), Staphylococcus aureus, and Streptococcus pyogenes. In some embodiments, the gram-positive bacterium is selected from methicillin-sensitive Staphylococcus aureus (MSSA), and methicillin-resistant Staphylococcus aureus (MRSA), Staphylococcus aureus, Staphylococcus epidermis and other coagulase-negative staphylococci, Streptococcus pyogenes, Streptococcus pneumoniae, Streptococcus agalactiae, and Enterococcus. In some embodiments, the gram-positive bacterium is selected from Staphylococcus spp, Streptococci, Enterococcus spp, Leuconostoc spp, Corynebacterium spp, Arcanobacteria spp, Trueperella spp, Rhodococcus spp, Bacillus spp, Anaerobic Cocci, Anaerobic Gram-Positive Nonsporulating Bacilli, Actinomyces spp, Clostridium spp, Nocardia spp, Erysipelothrix spp, Listeria spp, Kytococcus spp, Mycoplasma spp, Ureaplasma spp, and Mycobacterium spp.

In some embodiments, the bacterial infection is caused by a Gram-negative bacterium (e.g., Acinetobacter, Actinobacillus, Aeromonas, Anaplasma, Arcobacter, Avibacterium, Bacteroides, Bartonella, Bordetella, Borrelia, Brachyspira, Brucella, Campylobacter, Capnocytophaga, Chlamydia, Chlamydophila, Chryseobacterium, Coxiella, Cytophaga, Dichelobacter, Edwardsiella, Ehrlichia, Escherichia, Flavobacterium, Francisella, Fusobacterium, Gallibacterium, Haemophilus, Histophilus, Klebsiella, Lawsonia, Leptospira, Mannheimia, Megasphaera, Moraxella, Neorickettsia, Nicoletella, Ornithobacterium, Pasteurella, Photobacterium, Piscichlamydia, Piscirickettsia, Porphyromonas, Prevotella, Proteus, Pseudomonas, Rickettsia, Riemerella, Salmonella, Streptobacillus, Tenacibaculum, Vibrio, or Yersinia).

In specific embodiments, the Gram-negative bacterium is Pseudomonas aeruginosa.

In certain embodiments, the bacterial infection is caused by an antibiotic-resistant bacterium (e.g., antibiotic-resistant Pseudomonas aeruginosa).

In certain embodiments, the pharmaceutical compositions, DNAzymes, nucleic acids, or vectors described herein can be administered in conjunction with any other conventional antibacterial treatment, such as, for example, an antibiotic (e.g., penicillin, methicillin, cefoxitin, carbapenem, imipenem, and meropenem). These treatments may be applied as necessary and/or as indicated and may occur before, concurrent with or after administration of the pharmaceutical compositions, DNAzymes, nucleic acids, vectors, dosage forms, or kits described herein.

In certain embodiments, the method comprises the administration of multiple doses of the DNAzyme, nucleic acid, or vector. Separate administrations can include any number of two or more administrations (e.g., doses), including 2, 3, 4, 5, 6, 7, 8, 9, 10, 11, 12, 13, 14, 15, 16, 20, 21, 22, 23, 24, or 25 administrations. In some embodiments, at least 8, 9, 10, 11, 12, 13, 14, or 15 administrations are included. One skilled in the art can readily determine the number of administrations to perform, or the desirability of performing one or more additional administrations, according to methods known in the art for monitoring therapeutic methods and other monitoring methods provided herein. Accordingly, the methods provided herein include methods of providing to the subject one or more administrations of a DNAzyme, a nucleic acid, a vector and/or a pharmaceutical composition described herein, where the number of administrations can be determined by monitoring the subject, and based on the results of the monitoring, determining whether or not to provide one or more additional administrations. Deciding on whether or not to provide one or more additional administrations can be based on a variety of monitoring results, including, but not limited to, indication of bacterial growth or inhibition of bacterial growth, cleavage of murG RNA transcripts, expression level (e.g., RNA transcript and/or protein level) of murG, inhibition of bacterial cell wall biosynthesis, sensitization of antibiotic resistance, the subject's bacterial titer, the overall health of the subject and/or the weight of the subject.

Examples of routes of administration include oral administration, rectal administration, topical administration, inhalation (nasal) or injection. Administration by injection includes intravenous (IV), intraperitoneal, intranasal, intraarterial, intravesicle, intraocular, transdermal intralesional, intramuscular (IM), and subcutaneous (SC) administration. The compositions described herein can be administered in any form by any effective route, including but not limited to oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In some embodiments, the DNAzymes, nucleic acids, vectors, or pharmaceutical compositions described herein are administered orally, rectally, topically, intravesically, by injection into or adjacent to a draining lymph node, intravenously, by inhalation or aerosol, or subcutaneously. In some embodiments, the administration is parenteral administration (e.g., subcutaneous administration).

In some aspects, provided herein are methods of inhibiting bacterial growth or replication, inducing death of a bacterial cell, reducing the amount of a biofilm, and/or enhancing effectiveness of an antibiotic, comprising contacting the bacterial cell with a DNAzyme, a nucleic acid, or a vector described herein.

In some aspects, provided herein are methods of inhibiting cell wall biosynthesis of a bacterial cell, comprising contacting the bacterial cell with a DNAzyme, a nucleic acid, or a vector described herein.

In some aspects, provided herein are methods of cleaving a murG RNA transcript comprising contacting the murG RNA transcript with a DNAzyme described herein.

It is appreciated that certain features of the DNAzymes and uses thereof disclosed herein, which are, for clarity, described in the context of separate embodiments, may also be provided in combination in a single embodiment. Conversely, various features of the DNAzymes and uses thereof disclosed herein, which are, for brevity, described in the context of a single embodiment, may also be provided separately or in any suitable subcombination or as suitable in any other described embodiment of the DNAzymes and uses thereof disclosed herein. Certain features described in the context of various embodiments are not to be considered essential features of those embodiments, unless the embodiment is inoperative without those elements.

Various embodiments and aspects of the DNAzymes and uses thereof disclosed herein, as delineated hereinabove and as claimed in the claims section below find experimental support in the following examples.

### EXAMPLES

### Materials and Methods

Oligonucleotides. DNA oligonucleotides, including any modification, were custom ordered from Integrated DNA Technologies (IDT), reconstituted to 100 μM with ultrapure, DNase/RNase free water (Biological Industries, Israel), and stored at −20° C.

Bacterial strains. Multi-drug resistant Pseudomonas aeruginosa (MDR-PA, ATCC® BAA-3105™) were purchased from ATCC, streaked onto Luria broth (LB) agar plates (HyLabs, Israel) and grown for 24 h at 37° C. A single colony from each strain was selected and frozen in LB supplemented with 30% glycerol (HyLabs, Israel) and stored at −80° C. for all assays.

Flow cytometry. Flow cytometry was performed on a Becton-Dickinson Accuri C6 Plus cytometer equipped with 488 nm solid-state laser and a 640 nm diode laser or on a Sony ID7000M′ spectral cell analyzer. Data was analyzed using Kaluza Analysis 2.1 software using a C6 import module.

Intracellular DNAzyme quantification from bacterial cells. Bacterial strains were streaked from frozen LB+glycerol stocks onto LB agar plates and incubated for 24 h at 37° C. Single colonies were grown overnight at 37° C. in 3 ml of LB. Cultures were diluted to OD 0.01 in 2 ml LB+32 μg/ml meropenem, and DNAzyme were added to a final concentration of 1.25 μM. Cultures were incubated at 37° C. with continuous shaking for 4 hours. Cells were harvested after 4 hours, washed twice with 1×Dulbecco's Phosphate Buffered Saline, and treated with DNase I (New England Biolabs) for 5 min in 1×PBS at room temperature, then washed again with 1×PBS. For intracellular extraction of the DNAzymes, cells were incubated for 10 minutes at 95° C., and supernatants were collected for quantification of intracellular DNAzymes. Intracellular levels of DNAzymes were determined by quantitative polymerase chain reaction (qPCR), with primers designed using NCBI primer-BLAST. qPCR analysis was performed using the iTaq Universal SYBR Green Supermix (Bio-Rad), in a CFX96 system by the following program: 95° C. for 3 min, 39 cycles of 95° C. for 10 s and 55° C. for 30 s. Melt curves were generated for each sample by heating PCR amplicons from 65° C. to 95° C. with a gradual increase of 0.5° C./0.5 s.

To measure intracellular DNAzyme content of biofilm cells, bacterial cultures were grown in TSB for 24 hours at 37° C. without shaking, biofilm cells were isolated from non-adhered cells, and 1.25 μM fluorescent DNAzymes were added for two hours. Non-adherent cells were removed, biofilms were washed, and biofilm cells were extracted using mild sonication (2-4 pulses of 5 seconds, amplitude 30%). For measurements of intracellular fluorescent DNAzymes of bacterial cells colonizing lung tissue, bacterial cultures were grown with lung tissue samples in DMEM with meropenem for 24 hours at 30° C. with 5% CO₂ without shaking. After 24 hours, fluorescent DNAzyme were added, and samples and surrounding media were incubated at room temperature for an additional 4 hours. Cells colonizing the lung tissue were extracted by moderate sonication (2-4 pulses of 5 second, amplitude 30% per sample).

From all samples, cells were harvested every 30 min, washed twice with 1× Dulbecco's Phosphate Buffered Saline (PBS, Biological Industries) and were treated with DNase I (New England Biolabs) for 5 min in 1×PBS at room temperature and washed again with 1×PBS. Data were collected from −50,000 cells per time point using the FL-4 laser.

Growth assays. Bacterial strains were streaked from frozen LB+glycerol stocks onto LB agar plates and incubated for 24 h at 37° C. Single colonies were grown overnight at 37° C. in 3 ml of LB. Cultures were diluted to an initial OD of 0.01 in 2 ml LB+32 μg/ml meropenem and DNAzyme were added to a final concentration of 1.25 μM. Cultures were incubated at 37° C. with continuous shaking and the optical density OD₆₀₀ was recorded every 30 min using Ultrospec 10 (Biochrom). Viability was tested by serial dilutions of the bacterial suspensions, and spots were plated on LB plates. Plates were incubated at 37° C., and CFU were counted on the next day, to determine culture viability.

Biofilm assays. Bacterial strains were streaked from frozen LB+glycerol stocks onto LB agar plates and incubated for 24 h at 37° C. Single colonies were grown overnight at 37° C. in 3 ml of LB. Cultures were diluted 1:100 in TSB in a 96-well plate, and DNAzymes were added to a final concentration of 2.5 μM. Cultures were incubated at 37° C. without shaking for 16 hours. Non-adherent cells were removed, and biofilms were washed with PBS and either stained with crystal violet (0.1%), or biofilm cells were extracted using mild sonication (2-4 pulses of 5 seconds, amplitude 30%) for CFU determination. CFU was measured using serial dilutions of the bacterial suspensions and spots were plated on LB plates. CFU was determined as above.

E-test assay. The CLSI M100 guidelines for antimicrobial susceptibility testing disk were followed with slight modifications. Bacterial strains were streaked from frozen LB+glycerol stocks onto LB agar plates and incubated for 24 h at 37° C. Single colonies were grown overnight at 37° C. in 3 ml of LB. Colonies were suspended in 1 ml PBS and diluted to OD₆₀₀ 0.05 and were spread onto 15 ml Muller-Hinton plates supplemented with 50 M of the indicated DNAzyme. A single Meropenem E-test strip (Oxoid, UK) was placed in the center of each plate, plates were then incubated at 37° C. for 24 h, and each plate was photographed individually. Determination of MIC₉₀ analysis was performed using visual observation.

Bacterial CFU was tested by serial dilutions of the bacterial suspensions, and spots were plated on LB plates. The plates were incubated at 37° C. and on the next day CFU was counted to determine bacterial counts.

Ex vivo. Lungs were harvested from 5-10 mice (C57BL/6, 6-8 weeks old) and placed in petri dishes containing DMEM 5% FCS. The tissue was divided into circular samples 3 mm in diameter with a biopsy punch and transferred to poly-propanol tubes with 3 ml DMEM. DMEM contained meropenem and/or DNAzymes, as indicated. To each respective tube 10 μl of mid-logarithmic bacterial culture was added, with 3 technical repeats for each condition. Tubes were incubated at 37° C. for 24 hours and washed twice with PBS. For CFU of bacteria infecting ex vivo tissue culture, individual samples or their growth media were collected, samples were re-suspended in 1 ml PBS, and free-living bacteria were pelleted and re-suspended in PBS in the same volume. Cells colonizing the lung tissue were extracted by moderate sonication (2-4 pulses of 5 sec, amplitude 30% per samples). In all cases, to determine the number of viable cells, samples were serially diluted in PBS, plated on LB plates, and colonies were counted after incubation at 37° C. overnight.

### Results

### Example 1—Exemplary Structure of a DNAzyme

An exemplary structure for a 10-23 type DNAzyme is illustrated in FIG. 1. DNAzyme are nucleic acids that bind to and cleave RNA targets. In general, the structure of the DNAzyme included, in 5′ to 3′ order: (i) a first substrate-binding domain comprising a sequence that base pairs with a first region of a RNA transcript; (ii) a DNAzyme catalytic core; and (iii) a second substrate-binding domain comprising a sequence that base pairs with a second region of the RNA transcript positioned 5′ to the first region of the RNA transcript. Upon binding of the DNAzyme to the RNA transcript, the DNAzyme catalytic core cleaves the RNA transcript at a position between the first and second region of the RNA transcript.

### Example 2—Uptake of DNAzymes into Pseudomonas Aeruginosa

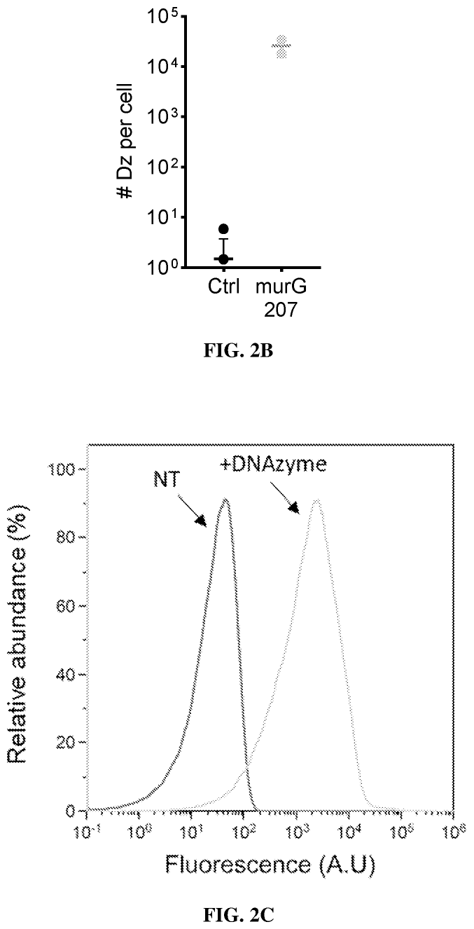

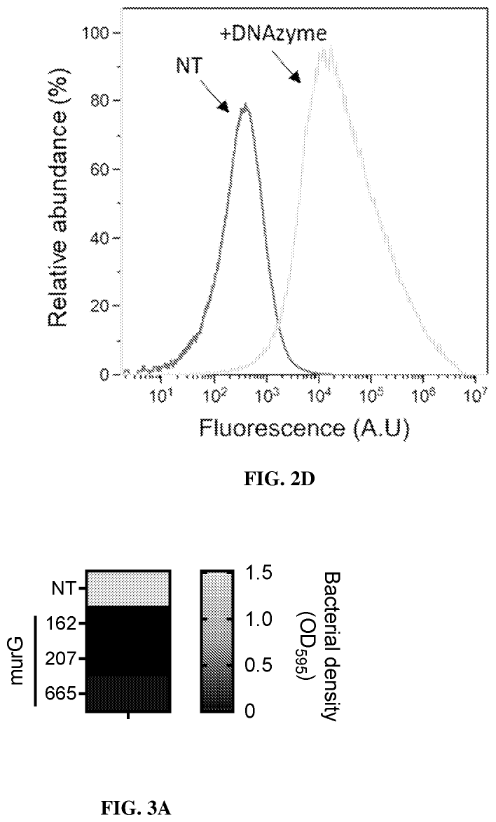

The first obstacle in using DNAzymes as antibacterial agents is efficient delivery of DNAzymes into bacterial cells via the cell wall. Additionally, the outer membrane of a gram-negative bacteria (which is absent in gram-positive bacteria) incurs resistance to a wide range of antibiotics due to its hydrophobic nature, which physically blocks the diffusion of some antibiotics. DNAzymes that bind and cleave murG were designed. Conjugation of cholesterol to DNAzymes directed to murG RNA transcripts facilitated the uptake of DNAzymes into Pseudomonas aeruginosa strain ATCC® BAA-3105™ grown in media, as shown in FIGS. 2A-2B. murG-207 DNAzyme is one embodiment of a DNAzyme targeting a transcript encoding a cell wall synthesis enzyme and was used in this study. The average copy number was over 10,000 DNAzymes per cell, indicating that the DNAzyme is capable of entering the bacteria.

To study the uptake of the described DNAzymes by bacteria in biofilms, cells were grown in TSB for 24 hours under conditions favorable to biofilm formation. Biofilm cells were separated from non-adhered cells incubated with fluorescent DNAzyme for 2 hours and analyzed by flow cytometry. FIG. 2C shows efficient uptake of fluorescent DNAzyme in bacterial cells residing in the biofilm.

Next, an ex vivo organ culture system was used to more closely recapitulate a biofilm in the 3D milieu of solid tissue, including various cell types and host extracellular matrix components. Mouse healthy naïve lung tissue samples were harvested by punch biopsy and cultured in an ex vivo culture system. The original topography of the lung tissue was maintained and host tissue viability could be maintained for several days ex vivo. In this system, bacterial cells colonizing the sample were grown in DMEM medium with meropenem, to maintain the host tissue and selectivity for P. aeruginosa. Significant uptake of DNAzymes by bacterial cells colonizing the tissue was observed (FIG. 2D).

### Example 3—Sensitization of Resistant Pseudomonas Aeruginosa by DNAzymes Targeting murG

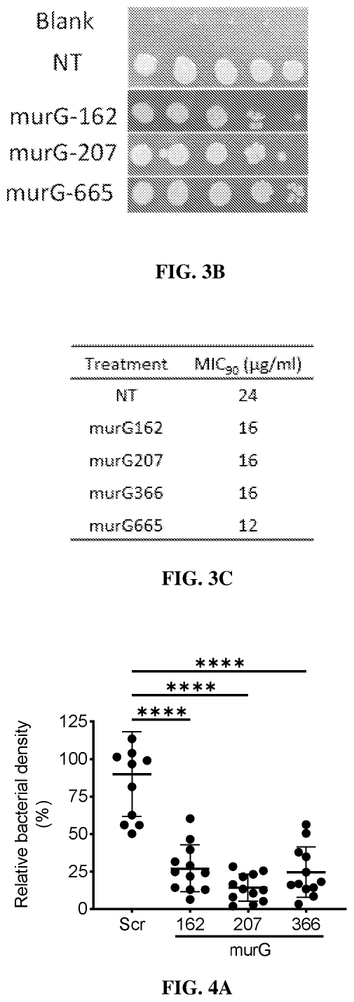

The objective was to test the ability of DNAzymes that target a transcript encoding a cell wall biosynthesis enzyme, to sensitize resistant bacterial strains to antibiotics. The resistant Pseudomonas aeruginosa strain ATCC® BAA-3105™ was incubated in LB+32 μg/ml meropenem, a meropenem concentration ordinarily sub-lethal to this strain, with or without DNAzymes targeting murG. Inhibition of bacterial growth was observed as measured by optical density (FIG. 3A) and the capacity to form colonies (FIG. 3B), demonstrating sensitization of resistant bacteria to antibiotics. DNAzymes also reduced MIC₉₀ levels of meropenem, as measured by e-test (FIG. 3C), confirming these findings.

### Example 4—Inhibition of Biofilm Formation by DNAzyme Treatment

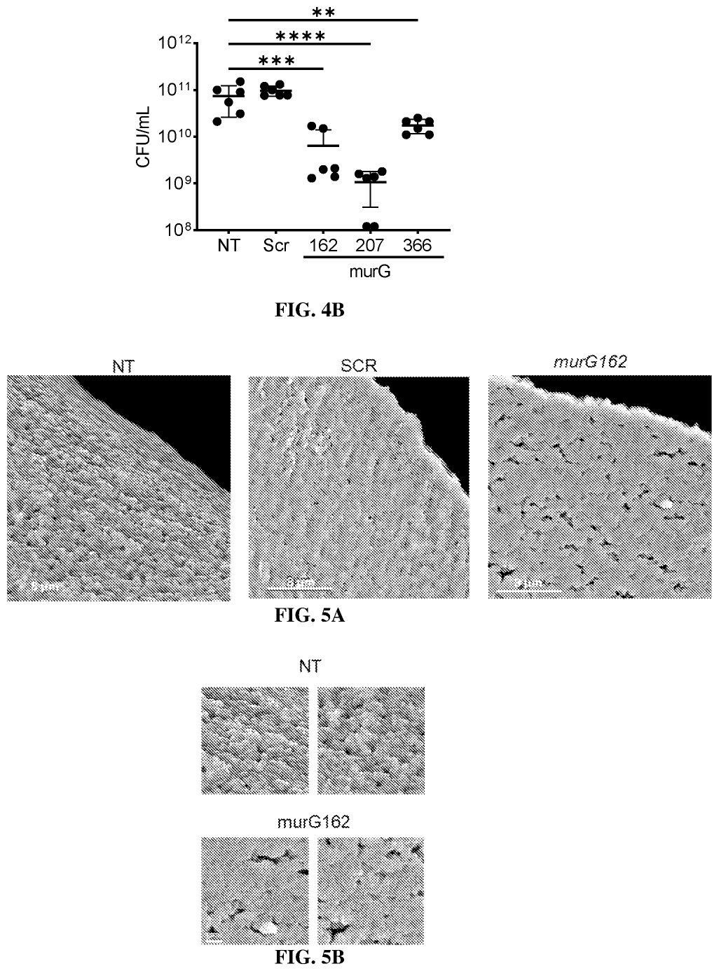

To measure the ability of DNAzymes that target a transcript encoding a cell wall biosynthesis enzyme, to affect biofilm formation, bacteria were grown in TSB on plates, under conditions that favor biofilm formation. Representative DNAzymes tested included murG-162, murG-207, and murG-366. Consistently, DNAzymes targeting murG showed biofilm inhibition and a significant reduction of viable cells within the biofilm (FIGS. 4A and 4B). DNAzymes unable to specifically target murG, Scr (SEQ ID NO: 44; scrambled nucleotide sequences in the 5′ and 3′ arm regions) did not show inhibition of biofilm formation or a reduction of viable cells.

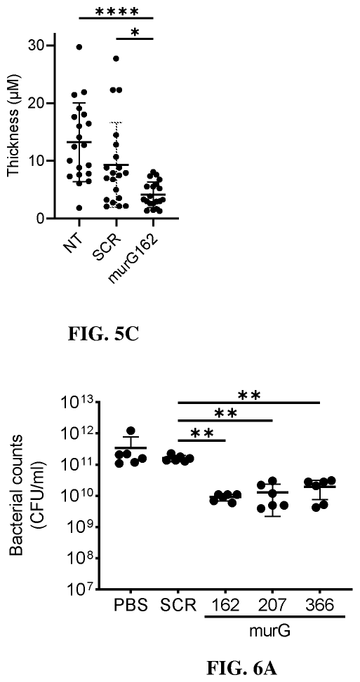

To determine the effects of the representative DNAzymes on macro- and microstructure of treated biofilms, 3D structures of treated and untreated biofilms were examined using scanning electron microscopy (SEM). Untreated biofilm (NT—No Treatment) and biofilm treated with scrambled DNA (SCR) had a typical thick and dense surface topography composed of aggregates of bacteria covered with exopolymers (left-panel and middle-panel), while biofilms treated with murG-162 had a smoother and less dense organization (right-panel), and with poorer surface coverage (FIG. 5A).

To study the effects of DNAzyme treatment on biofilm structure, 3D topographies of the biofilms were generated to compare multiple areas of the biofilm. The biofilms were dehydrated, resulting in a uniform loss of volume percentage. Untreated (NT) and scrambled DNA (SCR)-treated biofilms were composed of areas thicker than 10 μm, areas of about 10 μm, and significantly thinner areas (FIGS. 5B-C). Treatment with murG-162 significantly reduced the occurrence of areas thicker than 10 μm (FIG. 5C). Cells treated with murG-162 were more oval than untreated cells, had less clear borders between the clumped cells, and were more irregular and less covered in exopolymers than untreated biofilms (FIG. 5B) or biofilms treated with scrambled DNA (FIG. 5A). These results correlated the decrease in the average thickness of the treated biofilms to the loss of thick and structured areas in biofilms.

### Example 5—Effect of DNAzyme Treatment on Established Biofilms

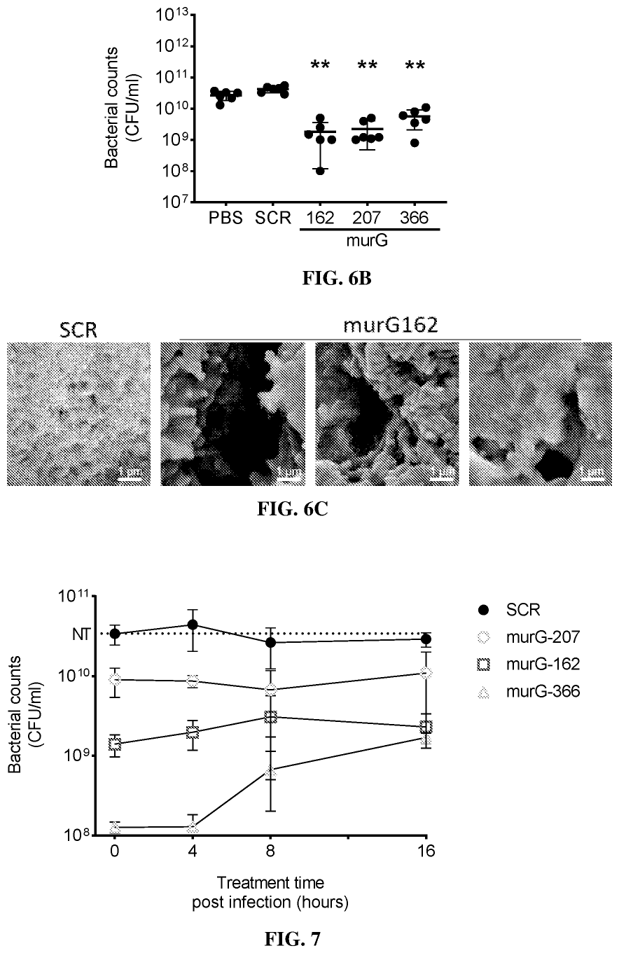

The effect of DNAzymes targeting murG on pre-established biofilms was studied by comparing the application of PBS, scrambled DNAzyme control (SCR), or indicated DNAzymes (162-murG, 207-murG, and 366-murG) on mature biofilms without or with the beta-lactam meropenem (FIGS. 6A-6B, respectively). The DNAzymes significantly reduced biofilm biomasses (˜1 log) and potentiated the effect of meropenem.

These findings were confirmed by SEM. Biofilms treated with sub-lethal concentrations of meropenem with control SCR DNAzyme were thinner (no thicker than 5 μm) and had more texture. Similar results were observed with biofilms treated with sub-lethal concentrations of meropenem alone (data not shown). FIG. 6C presents an example of these findings, wherein SCR DNAzyme treated samples were compared to biofilms treated with murG162+meropenem. The latter group also exhibited a high frequency of uncovered surface patches that were either not colonized by bacteria or cleared by microbial lysis.

### Example 6—Effect of DNAzyme Treatment on Bacteria in Lung Tissue Samples at Different Colonization Stages

Next, an ex-vivo model was used to mimic the P. aeruginosa infection, at various stages of lung colonization. Mouse lung tissue samples (naïve and healthy tissue) were harvested by punch biopsy and incubated DMEM medium+meropenem, to maintain selection for MDR-P. aeruginosa strains. DNAzymes (SCR, murG-207, murG-162, murG-366, were added at different stages of infection, and the amount of viable cells was measured after the infection cycle.

At the pre-colonization stage, the DNAzymes inhibited bacterial load by 0.5-2.5 logs (FIG. 7; time=0), indicating inhibition of adherence of the bacteria to lung tissue and/or successful colonization of the tissue.

At mid colonization (t=4 hours post infection) and after most of the bacteria were associated with the lung tissue (t=8 hours post infection), the activity of the DNAzymes was still significant, indicating that DNAzyme are still effective during lung colonization (FIG. 7).

After 16 h post infection, even as the number of tissue-associated bacteria declined due to tissue deterioration, the DNAzymes were still capable of decreasing bacterial load (FIG. 7; t=16).

1. A method of reducing an amount of a biofilm in a mammalian subject with a bacterial infection, said method comprising administering a composition comprising a DNAzyme targeting a murG RNA transcript, wherein said DNAzyme comprises (a) a first substrate-binding domain that is complementary to a first region of the transcript or partially complementary to the first region of said transcript; and (b) a second substrate-binding domain that is complementary to a second region of said transcript or partially complementary to the second region of said transcript; wherein
the first substrate-binding domain comprises nucleic acid sequence 5′-GATCAGGTG-3′ (SEQ ID NO: 7), and the second substrate-binding domain comprises nucleic acid sequence 5′-AACGGCAGG-3′ (SEQ ID NO: 8);
the first substrate-binding domain comprises the nucleic acid sequence 5′-CTTGACCAG-3′ (SEQ ID NO: 9), and the second substrate-binding domain comprises nucleic acid sequence 5′-GACTTCAGG-3′ (SEQ ID NO: 10);
the first substrate-binding domain comprises nucleic acid sequence 5′-ATCTCGGCA-3′ (SEQ ID NO: 11), and the second substrate-binding domain comprises nucleic acid sequence 5′-GCTGACGAC-3′ (SEQ ID NO: 12); or
the first substrate-binding domain comprises nucleic acid sequence 5′-GGCGTTCTG-3′ (SEQ ID NO: 31), and the second substrate-binding domain comprises nucleic acid sequence 5′-TCGTGGATC-3′ (SEQ ID NO: 32) wherein said administration reduced the amount of the biofilm is said mammalian subject.

2. A method of increasing antibiotic susceptibility in a mammalian subject, said method comprising administering a composition comprising a DNAzyme targeting a murG RNA transcript, wherein said DNAzyme comprises (a) a first substrate-binding domain that is complementary to a first region of the transcript or partially complementary to the first region of said transcript; and (b) a second substrate-binding domain that is complementary to a second region of said transcript or partially complementary to the second region of said transcript; wherein
the first substrate-binding domain comprises nucleic acid sequence 5′-GATCAGGTG-3′ (SEQ ID NO: 7), and the second substrate-binding domain comprises nucleic acid sequence 5′-AACGGCAGG-3′ (SEQ ID NO: 8);
the first substrate-binding domain comprises the nucleic acid sequence 5′-CTTGACCAG-3′ (SEQ ID NO: 9), and the second substrate-binding domain comprises nucleic acid sequence 5′-GACTTCAGG-3′ (SEQ ID NO: 10);
the first substrate-binding domain comprises nucleic acid sequence 5′-ATCTCGGCA-3′ (SEQ ID NO: 11), and the second substrate-binding domain comprises nucleic acid sequence 5′-GCTGACGAC-3′ (SEQ ID NO: 12); or
the first substrate-binding domain comprises nucleic acid sequence 5′-GGCGTTCTG-3′ (SEQ ID NO: 31), and the second substrate-binding domain comprises nucleic acid sequence 5′-TCGTGGATC-3′ (SEQ ID NO: 32),
wherein administration of said DNAzyme increases antibiotic susceptibility in said mammalian subject with a bacterial infection.

3. A method for inhibiting bacterial growth in a subject with a bacterial infection, said method comprising administering a composition comprising a DNAzyme targeting a murG RNA transcript, wherein said DNAzyme comprises (a) a first substrate-binding domain that is complementary to a first region of the transcript or partially complementary to the first region of said transcript; and (b) a second substrate-binding domain that is complementary to a second region of said transcript or partially complementary to the second region of said transcript; wherein
the first substrate-binding domain comprises nucleic acid sequence 5′-GATCAGGTG-3′ (SEQ ID NO: 7), and the second substrate-binding domain comprises nucleic acid sequence 5′-AACGGCAGG-3′ (SEQ ID NO: 8);
the first substrate-binding domain comprises the nucleic acid sequence 5′-CTTGACCAG-3′ (SEQ ID NO: 9), and the second substrate-binding domain comprises nucleic acid sequence 5′-GACTTCAGG-3′ (SEQ ID NO: 10);
the first substrate-binding domain comprises nucleic acid sequence 5′-ATCTCGGCA-3′ (SEQ ID NO: 11), and the second substrate-binding domain comprises nucleic acid sequence 5′-GCTGACGAC-3′ (SEQ ID NO: 12); or
the first substrate-binding domain comprises nucleic acid sequence 5′-GGCGTTCTG-3′ (SEQ ID NO: 31), and the second substrate-binding domain comprises nucleic acid sequence 5′-TCGTGGATC-3′ (SEQ ID NO: 32), wherein said administration inhibits the bacterial growth in said subject with a bacterial infection.

4. The method according to claim 1, wherein the DNAzyme catalytic core is a 10-23 catalytic core, an 8-17 catalytic core, a E1111 catalytic core, a E2112 catalytic core, a E5112 catalytic core, or a bipartite catalytic core

5. (canceled)

6. (canceled)

7. (canceled)

8. (canceled)

9. (canceled)

10. (canceled)

11. (canceled)

12. (canceled)

13. (canceled)

14. (canceled)

15. The method according to claim 1, wherein the bacterial infection is an infection with an antibiotic-resistant bacterium.

16. The method according to claim 1, wherein the DNAzyme comprises a chemical modification selected from a base modification, a sugar modification, and an internucleotide linkage modification.

17. The method according to claim 1, wherein the DNAzyme is linked to a cell penetration-enhancing moiety, wherein optionally the cell penetration-enhancing moiety is cholesterol.

18. (canceled)

19. (canceled)

20. The method according to claim 1, wherein said composition further comprises an antibiotic, wherein said antibiotic is selected from penicillin, methicillin, cefoxitin, carbapenem, imipenem, and meropenem.

21. (canceled)

22. The method according to claim 1, wherein the mammalian subject is a human.

23. (canceled)

24. The method according to claim 1, wherein the nucleotide sequence of said DNAzyme comprises SEQ ID NO: 13, SEQ ID NO: 19, SEQ ID NO: 25, or SEQ ID NO: 33.

25. The method according to claim 2, wherein the DNAzyme catalytic core is a 10-23 catalytic core, an 8-17 catalytic core, a E1111 catalytic core, a E2112 catalytic core, a E5112 catalytic core, or a bipartite catalytic core

26. The method according to claim 2, wherein the bacterial infection is an infection with an antibiotic-resistant bacterium.

27. The method according to claim 2, wherein the DNAzyme is linked to a cell penetration-enhancing moiety, wherein optionally the cell penetration-enhancing moiety is cholesterol.

28. The method according to claim 2, wherein said composition further comprises an antibiotic, wherein said antibiotic is selected from penicillin, methicillin, cefoxitin, carbapenem, imipenem, and meropenem.

29. The method according to claim 2, wherein the mammalian subject is a human.

30. The method according to claim 2, wherein the nucleotide sequence of said DNAzyme comprises SEQ ID NO: 13, SEQ ID NO: 19, SEQ ID NO: 25, or SEQ ID NO: 33.

31. The method according to claim 3, wherein the DNAzyme catalytic core is a 10-23 catalytic core, an 8-17 catalytic core, a E1111 catalytic core, a E2112 catalytic core, a E5112 catalytic core, or a bipartite catalytic core

32. The method according to claim 3, wherein the bacterial infection is an infection with an antibiotic-resistant bacterium.

33. The method according to claim 3, wherein the DNAzyme is linked to a cell penetration-enhancing moiety, wherein optionally the cell penetration-enhancing moiety is cholesterol.

34. The method according to claim 3, wherein said composition further comprises an antibiotic, wherein said antibiotic is selected from penicillin, methicillin, cefoxitin, carbapenem, imipenem, and meropenem.

35. The method according to claim 3, wherein the mammalian subject is a human.

36. The method according to claim 3, wherein the nucleotide sequence of said DNAzyme comprises SEQ ID NO: 13, SEQ ID NO: 19, SEQ ID NO: 25, or SEQ ID NO: 33.
